(19) United States
(12) Patent Application Publication
(10) Pub. No.: US 2026/0325292 A1
(43) Pub. Date: Oct. 22, 2026

(54) PROSTHETIC HEART VALVES

(71) Applicant: Edwards Lifesciences Corporation, Irvine, CA (US)

(72) Inventors: Tamir S. Levi, Zikhron Yaakov (IL); Roy Shitrit, Zichron Yaakov (IL); David Maimon, Atlit (IL); Michael Bukin, Pardes Hanna (IL); Bich Hoang Pham, Irvine, CA (US); Christine Uyen Dang, Garden Grove, CA (US)

(73) Assignee: Edwards Lifesciences Corporation, Irvine, CA (US)

(21) Appl. No.: 19/468,819

(22) Filed: Feb. 3, 2026

Related U.S. Application Data
(63) Continuation of application No. PCT/US2024/041077, filed on Aug. 6, 2024.
(60) Provisional application No. 63/531,067, filed on Aug. 7, 2023.

(51) Int. Cl. A61F 2/24

(52) U.S. Cl. CPC A61F 2/2418; A61F 2210/0076; A61F 2220/0075

(57) ABSTRACT

Barrier layers for a prosthetic heart valve are disclosed. As one example, a prosthetic heart valve can include an annular frame having an inflow end and an outflow end, a plurality of leaflets disposed within the annular frame, each leaflet comprising a cusp edge portion that is positioned towards the inflow end of the frame, a skirt coupled to the annular frame, and a barrier layer coupled to each leaflet and comprising a first end and a second end, wherein the first end is coupled to the cusp edge portion and the second end is coupled to the skirt, wherein the barrier layer at least partially extends around the inflow end of the annular frame.

### CROSS-REFERENCE TO RELATED APPLICATIONS

This application is a continuation of PCT Application No. PCT/US2024/041077, filed Aug. 6, 2024, which claims the benefit of U.S. Patent Application No. 63/531,067, filed Aug. 7, 2023. The prior applications are incorporated by reference herein in their entireties.

### FIELD

The present disclosure relates to prosthetic heart valves, and in particular to barrier layers for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are prosthetic heart valves, delivery apparatus, and methods for implanting prosthetic heart valves. The disclosed prosthetic heart valves, delivery apparatus, and methods can, for example, provide improved laminar flow at the inflow ends of prosthetic heart valve, among other things. As such, the devices and methods disclosed herein can, among other things, provide improved prosthetic heart valves.

A prosthetic heart valve can comprise a frame and a valve structure coupled to the frame. In addition to these components, a prosthetic heart valve can further comprise one or more of the components disclosed herein.

In some examples, a prosthetic heart valve can comprise a barrier layer configured to improve laminar flow at an inflow end of the valve.

In some examples, a prosthetic heart valve can comprise a barrier layer configured to prevent cell growth (e.g., smooth muscle cells, etc.) from migrating from an exterior of the valve towards an interior of the valve.

In some examples, a prosthetic heart valve can comprise a frame and a barrier layer coupled to an inflow end of the frame, wherein a first portion of the barrier layer is positioned radially inwards of the frame and a second portion is positioned radially outwards of the frame.

In some examples, a prosthetic heart valve can comprise a frame, a leaflet disposed within the frame, and a barrier layer coupled to the frame, wherein a portion of the leaflet is positioned between the barrier layer and the frame.

In some examples, a prosthetic heart valve comprises an annular frame having an inflow end and an outflow end; a plurality of leaflets disposed within the annular frame, each leaflet comprising a cusp edge portion that is positioned towards the inflow end of the frame; a skirt coupled to the annular frame; and a barrier layer coupled to each leaflet and comprising a first end and a second end, wherein the first end is coupled to the cusp edge portion and the second end is coupled to the skirt, wherein the barrier layer at least partially extends around the inflow end of the annular frame.

In some examples, a prosthetic heart valve comprises an annular frame; a plurality of leaflets disposed within the annular frame, each leaflet comprising a cusp edge portion comprising a suture line and a free edge; and a barrier layer coupled to a cusp edge portion of each leaflet at the suture line, wherein the free edge of the leaflet is positioned between the barrier layer and the frame.

In some examples, a prosthetic heart valve comprises an annular frame having an inflow end and an outflow end; a plurality of leaflets disposed within the annular frame, each leaflet comprising a cusp edge that is positioned towards the inflow end of the frame; and a barrier layer disposed within the annular frame, wherein at least a portion of the cusp edge of the leaflet is positioned between the barrier layer and the frame.

In some examples, a prosthetic heart valve comprises an annular frame having an inflow end, an outflow end, and a central longitudinal axis extending from the inflow end to the outflow end; a plurality of leaflets disposed within the annular frame, each leaflet comprising a cusp edge that is positioned towards the inflow end of the frame; a first barrier layer coupled to the frame, wherein the first barrier layer is radially positioned between the leaflets and the frame; and a second barrier layer disposed within the annular frame and comprising a first end and a second end, wherein at least a portion of the cusp edge of the leaflet is radially positioned between the first end of the second barrier layer and the frame, and wherein the second end of the second barrier layer is positioned towards the inflow end of the annular frame.

In some examples, a prosthetic heart valve comprises one or more of the components recited in Examples 1–20 and 24–44 below.

In some examples, a leaflet construct can comprise a plurality of leaflets configured to regulate the flow of blood in one direction.

In some examples, a leaflet construct can comprise a plurality of leaflets and one or more barrier layers coupled to the plurality of leaflets.

In some examples, a leaflet construct can comprise a plurality of leaflets, each leaflet comprising a cusp edge portion; a barrier layer coupled to the cusp edge portion of each leaflet, the barrier layer comprising a polymeric layer that is resistant to tissue ingrowth and a suture extending through the polymeric layer.

In some examples, a leaflet construct comprises one or more of the components recited in Examples 21–23 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a cross-sectional side view of the prosthetic heart valve of FIG. 1.

FIG. 3 is a cross-sectional side view of the prosthetic heart valve of FIG. 1 with a barrier layer at the inflow end of the valve.

FIG. 4 is a side view of a barrier layer, according to one example.

FIG. 5 is a partial side view of a prosthetic heart valve, according to one example, in a flattened configuration, that includes the barrier layer of FIG. 4 in a first configuration and shows one leaflet for purposes of illustration.

FIG. 6A is a cross-sectional view of the prosthetic heart valve of FIG. 5, taken along section 6A-6A shown in FIG. 5.

FIG. 6B is a cross-sectional view of the prosthetic heart valve of FIG. 5, taken along section 6B-6B shown in FIG. 5.

FIGS. 7A–7B are cross-sectional views of a prosthetic heart valve with the barrier layer of FIG. 4 in other configurations, according to one example, taken along a section similar to 6A-6A of FIG. 5.

FIG. 8 is a cross-sectional view of a prosthetic heart valve with the barrier layer of FIG. 4 in another configuration, according to one example, taken along a section similar to 6B-6B of FIG. 5.

FIG. 9 is a cross-sectional view of a prosthetic heart valve with the barrier layer of FIG. 4 in another configuration, according to one example, taken along a section similar to 6B-6B of FIG. 5.

FIGS. 10A–10B illustrate a barrier layer, according to one example.

FIGS. 11A–11B are cross-sectional views of a prosthetic heart valve with the barrier layer of FIGS. 10A–10B, according to one example, taken along a section similar to 6A-6A of FIG. 5.

FIG. 12A is a cross-sectional view of a prosthetic heart valve with a plurality of barrier layers, according to one example, taken along a section similar to 6B-6B of FIG. 5.

FIG. 12B is a partial side view of the barrier layers and leaflet of the prosthetic heart valve of FIG. 12A.

FIG. 13A is a cross-sectional view of a prosthetic heart valve with a plurality of barrier layers, according to one example, taken along a section similar to 6B-6B of FIG. 5.

FIG. 13B is a partial side view of the barrier layers and leaflet of the prosthetic heart valve of FIG. 13A.

FIG. 14 is a cross-sectional view of a prosthetic heart valve with a plurality of barrier layers, according to one example, taken along a section similar to 6B-6B of FIG. 5.

FIG. 15 is a partial side view of the barrier layers and the frame of the prosthetic heart valve of FIG. 14.

FIGS. 16A and 16B are side views of the barrier layers of the prosthetic heart valve of FIG. 14, according to one example.

FIGS. 17A and 17B are side views of the barrier layers of the prosthetic heart valve of FIG. 14, according to one example.

FIG. 18 is a side view of a leaflet construct of the prosthetic heart valve of FIG. 14 in a laid flat configuration.

FIGS. 19A and 19B are side views of the leaflet construct of FIG. 18, with a single-passed pledget line and a double-passed pledget line, respectively.

FIG. 20 is a side view of the prosthetic heart valve of FIG. 14 in a partially assembled state, illustrating fasteners coupling the first barrier layer to the frame.

FIG. 21 is a detailed view of fasteners coupling the first barrier layer to the frame of the prosthetic heart valve of FIG. 14.

FIGS. 22A–22C are detailed perspective views illustrating a hammock technique for coupling the frame of the prosthetic heart valve of FIG. 14 to the pledget line.

FIGS. 23A and 23B are detailed perspective views illustrating techniques for coupling the first barrier layer to the leaflet construct within the frame of the prosthetic heart valve of FIG. 14, along a side portion of the first barrier layer.

FIG. 24 is a detailed perspective view illustrating a technique for coupling the first barrier layer to the leaflet construct within the frame of the prosthetic heart valve of FIG. 14, along an inflow portion of the first barrier layer.

FIG. 25A is a side view of the prosthetic heart valve of FIG. 14 in a partially assembled state, illustrating fasteners coupling the first barrier layer to the frame and coupling the first barrier layer to the leaflet construct.

FIG. 25B is a side view of the prosthetic heart valve of FIG. 14 in a partially assembled state, illustrating fasteners coupling the first barrier layer to the leaflet construct.

FIG. 26 is a perspective view of an interior of the prosthetic heart valve of FIG. 14, illustrating a double-passed pledget line.

FIG. 27 is a side view of the prosthetic heart valve of FIG. 14 in a partially assembled state, illustrating fasteners coupling the first barrier layer to the leaflet construct.

FIG. 28 is a perspective view of an interior of the prosthetic heart valve of FIG. 14 radially between the first barrier layer and the leaflet.

FIG. 29 is a side view of the prosthetic heart valve of FIG. 14 in a partially assembled state illustrating a technique for coupling an outer skirt to the frame.

FIG. 30 is a side view of the prosthetic heart valve of FIG. 14 in an assembled state.

FIG. 31 is a cross-sectional view of a prosthetic heart valve with a plurality of barrier layers, according to one example, taken along a section similar to 6B-6B of FIG. 5.

FIG. 32 is a cross-sectional view of a prosthetic heart valve with a plurality of barrier layers, according to one example, taken along a section similar to 6B-6B of FIG. 5.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient’s body), while distal motion of the device is motion of the device away from the user and toward the implantation site (e.g., into the patient’s body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient’s vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

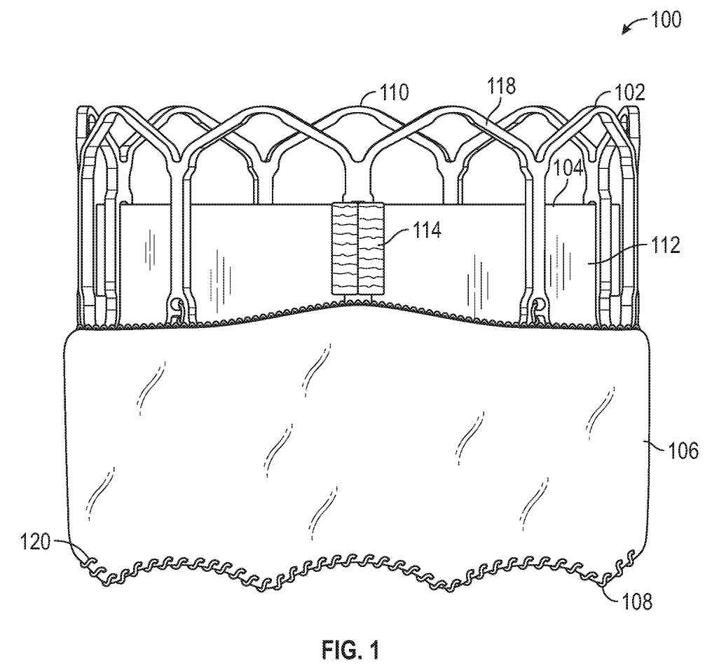

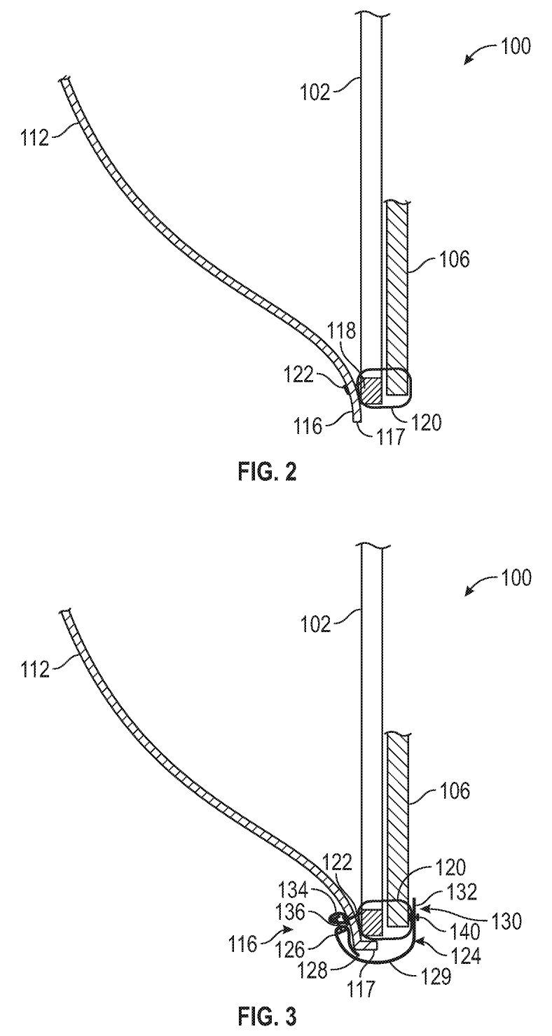

As introduced above, some prosthetic heart valves can include an outer skirt disposed around an outer surface of an annular frame of the prosthetic heart valve. The outer skirt can be configured to form a seal against native tissue upon implantation of the prosthetic heart valve, thereby reducing paravalvular leakage (PVL) between the prosthetic heart valve and the native anatomy (e.g., a native heart valve annulus and/or the native leaflets). FIGS. 1 and 2 illustrate an exemplary prosthetic device (e.g., prosthetic heart valve) comprising a frame, leaflets secured on an inside of the frame, and an outer skirt disposed around an outer surface of the frame. The prosthetic device can be advanced through a patient’s vasculature, such as to a native heart valve, by a delivery apparatus.

In some examples, a prosthetic heart valve can include one or more barrier layers, such as one or more of the barrier layers depicted in FIGS. 3–32. A barrier layer can be positioned at an inflow end of the prosthetic heart valve (e.g., at an inflow end of each leaflet, etc.). In some examples, the barrier layer can be configured to improve laminar flow at the inflow end of the valve (e.g., at an inflow end of the leaflet, etc.). In some examples, the barrier layer can comprise a thromboresistant material. In some examples, the barrier layer can be configured to prevent cell growth (e.g., smooth muscle cells, etc.) from migrating from an exterior of the valve towards an interior of the valve. In some examples, multiple barrier layers can be used. As a result, the devices and methods disclosed herein can, among other things, improve prosthetic heart valves by enabling native tissue ingrowth on the exterior of the prosthetic valve and reducing native tissue ingrowth on the interior of the prosthetic valve, improving hemodynamics through the interior of the prosthetic valve, and/or while also reducing thrombus formation.

### Examples of the Disclosed Technology

FIG. 1 shows a prosthetic heart valve 100 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Patent Application Publication No. 2017/0231756, which is incorporated by reference herein. In another example, the disclosed prosthetic valves can be implanted within a docking device implanted within or at the native mitral valve, such as disclosed in International Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Patent Application Publication No. 2019/0000615, which is incorporated by reference herein.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102. In some examples, the prosthetic valve 100 can include an inner skirt disposed on an interior of the frame 102.

The valvular structure 104 can comprise a plurality of leaflets 112 (e.g., three leaflets, as shown in FIG. 1), collectively forming a leaflet structure, which can be arranged to collapse in a tricuspid arrangement, although in other examples there can be greater or fewer number of leaflets (e.g., one or more leaflets 112). The leaflets 112 can be secured to one another at their adjacent sides (e.g., commissure tabs) to form commissures 114 of the valvular structure 104. For example, each leaflet 112 can comprise opposing commissure tabs disposed on opposite sides of the leaflet 112 and a cusp edge portion 116 (FIG. 2) extending between the opposing commissure tabs at or adjacent an inflow edge 117 of each leaflet 112. The cusp edge portion 116 of the leaflets 112 can have an undulating, curved scalloped shape, and can be secured directly to the frame 102 (e.g., by sutures). However, in alternate examples, the cusp edge portions 116 can be secured to an inner skirt which is then secured to the frame 102. In some examples, the leaflets 112 can be formed of pericardial tissue (e.g., bovine pericardial tissue), biocompatible synthetic materials, or various other suitable natural or synthetic materials as known in the art and described in U.S. Patent No. 6,730,118, which is incorporated by reference herein.

In some examples, the outer skirt 106 can be an annular skirt. The skirt can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide, etc. In some examples, the skirt can comprise a non-textile or non-fabric material, such as a film made from any of a variety of polymeric materials, such as PTFE, PET, polypropylene, polyamide, polyether ether ketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources). In some examples, the skirt can comprise an elastic or stretchable material. Non-limiting examples of stretchable or elastic materials include polyethylene terephthalate (PET), polytetrafluoroethylene (PTFE), expanded PTFE (ePTFE), and the like. In some examples, the skirt can comprise one or more layers of one or more of the materials described herein.

The frame 102 can be radially compressible and expandable between a radially compressed configuration and a radially expanded configuration (the expanded configuration is shown in FIG. 1) and can comprise a plurality of interconnected struts 118.

The frame 102 can be made of any of various suitable plastically-expandable materials (e.g., stainless steel, etc.) or self-expanding materials (e.g., nickel titanium alloy (NiTi), such as nitinol) as known in the art. When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Fasteners or sutures, such as sutures comprising whip stitches 120, can be used to secure the outer skirt 106 to the struts 118 of the frame 102. As described above, the cusp edge portion 116 of the leaflets 112 can be coupled to the frame 102 and/or an inner skirt. As shown in FIG. 2, the cusp edge portions 116 can be secured to the frame 102 with fasteners, such as in-and-out stitches 122. In some examples, as depicted, the in-and-out stitches 122 can extend through the leaflet 112 and around one or more of the whip stitches 120 to secure the leaflet 112 to the frame 102. As shown in FIG. 2, in some examples, the in-and-out stitches 122 can be slightly spaced apart from the inflow edge 117 of the leaflets 112, such that free portions of the leaflets 112 between the stitches 122 and the inflow edges 117 can extend towards or beyond the inflow end 108 of the frame 102.

In some instances, it may be desirable to enclose or secure the free portions of the leaflets 112 within a layer of material (e.g., barrier layer 124) to create a smoother transition at the inflow edge 117 of the leaflet 112. FIG. 3 illustrates examples of a barrier layer 124 as coupled to the prosthetic heart valve 100 of FIGS. 1-2. As shown, the barrier layer 124 is coupled to the cusp edge portion 116 of the leaflet 112 and disposed around the inflow edge 117 of the leaflet 112. In this way, the barrier layer 124 can enclose or surround the inflow edge 117 of the leaflet 112, such that the inflow edge 117 is disposed between the barrier layer 124 and the frame 102.

The barrier layer 124 can include a first end portion 126 including a first edge 128 and a second end portion 130 including a second edge 132. The barrier layer 124 can also include an intermediate portion 129 between the first end portion 126 and the second end portion 130. As shown in FIG. 3, the first end portion 126 is positioned radially inwards of the frame 102 (e.g., within the frame 102) and is coupled, for example, to the cusp edge portion 116 of the leaflet 112. The second end portion 130 is positioned radially outside of the frame 102 and coupled, for example, to the outer skirt 106. In this way, the barrier layer 124 extends around the inflow end 108 of the frame 102 (e.g., from a position that is radially outwards of the frame 102 to a position that is radially inwards of the frame 102).

The end portions of the barrier layer 124 can be in a folded or unfolded configuration. For example, as shown in FIG. 3, the first end portion 126 is in a folded configuration and the second end portion 130 is in an unfolded configuration. In some examples, both end portions 126, 130 can be in a folded configuration. In some examples, both end portions 126, 130 can be in a folded configuration. In the illustrated example, the first end portion 126 can be folded into the folded configuration, such that the first edge 128 is positioned between the frame 102 and the intermediate portion 129 of the barrier layer 124. In this way, the first edge 128 is radially and/or axially inwards of the intermediate portion 129 of the barrier layer 124. In the illustrated example, the second end portion 130 of the barrier layer 124 is in the unfolded configuration, such that the second edge 132 extends towards the outflow end 110 of the valve 100. In some instances, having an end portion of the barrier layer 124 in the folded configuration can further improve laminar flow at the inflow end 108 of the prosthetic heart valve 100. In some instances, the folded configuration can also provide strain relief at the region where the leaflets 112, skirt 106, and/or barrier layer 124 are coupled together.

Fasteners or sutures, such as sutures comprising whip stitches 134 and/or in-and-out stitches 136, can be used to secure the first end portion 126 of the barrier layer 124 in a position that is radially inward of the frame 102 (e.g., to the leaflet 112). Specifically, in some examples, in-and-out stitches 136 can extend through the barrier layer 124. Whip stitches 134 can pass through the in-and-out stitches 136 of the barrier layer 124 and the in-and-out stitches 122 of the leaflet 112, as shown in FIG. 3, to secure the barrier layer 124 to the leaflet 112. Fasteners or sutures, such as sutures comprising whip stitches and/or in-and-out stitches 140, can be used to secure the second end portion 130 of the barrier layer 124 in a position that is radially outward of the frame 102 (e.g., to the outer skirt 106). Specifically, in some examples, in-and-out stitches 140 can extend through the barrier layer 124 and the whip stitches 120 that secure the outer skirt 106 to the frame 102, as shown in FIG. 3, to secure the barrier layer 124 to the outer skirt 106. Other configurations of fasteners or sutures (e.g., whip stitches, in-and-out stitches, other stitches, fasteners, etc.) can be used to couple the barrier layer 124 to the inflow end 108 of the prosthetic heart valve 100.

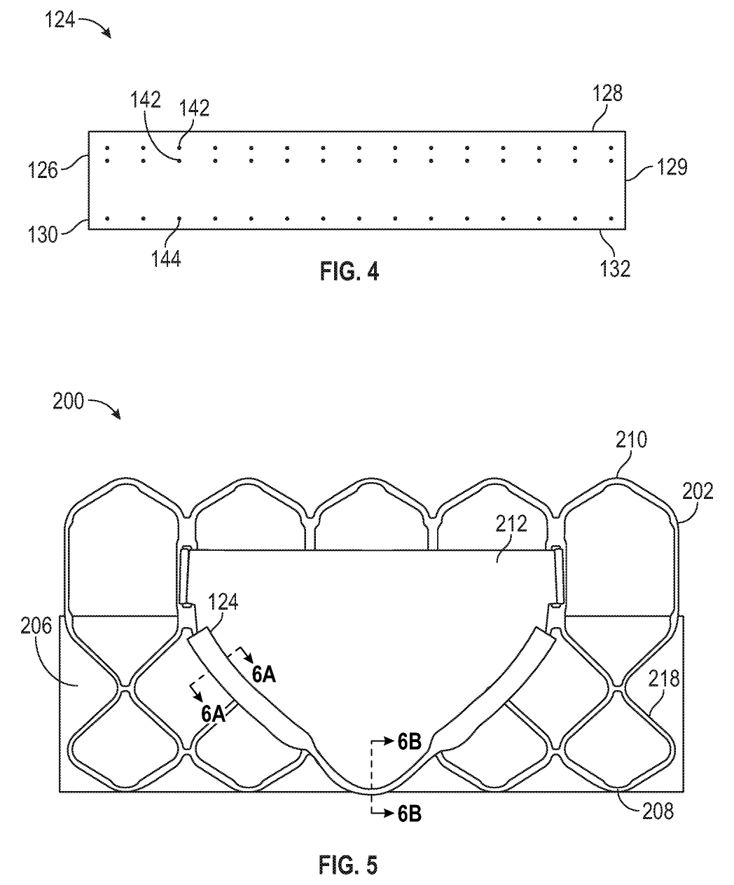

FIG. 4 illustrates barrier layer 124 in greater detail. As shown, the barrier layer 124 can comprise a plurality of first apertures 142 positioned along the length of the first end portion 126 and a plurality of second apertures 144 positioned along the length of the second end portion 130.The first apertures 142 and second apertures 144 can each comprise one or more rows of apertures. In some examples, as depicted, the first apertures 142 can comprise two rows of apertures that are spaced apart from each other (e.g., spaced apart in the direction between the first edge 128 and the second edge 132), and the second apertures 144 can comprise one row of apertures. In some instances, the number of rows of apertures can correspond to the folded or unfolded configuration of the end portion of the barrier layer 124 (e.g., one row for unfolded and two rows for folded, etc.). When the first end portion 126 of the barrier layer 124 is folded over into the folded configuration, the two rows of first apertures 142 can be aligned. In some examples, the whip stitches 134 and/or in-and-out stitches 136 can extend or pass through the first apertures 142, for example, to maintain the first end portion 126 in the folded configuration and couple the first end portion 126 to the leaflet 112. For an unfolded configuration, the whip stitches and/or in-and-out stitches 140 can extend or pass through the single row of second apertures 144 to secure the second end portion 130 of the barrier layer 124 outside of the frame 102 (e.g., to the outer skirt 106). In some examples, the barrier layer 124 can include additional apertures or openings for fasteners or sutures to couple the barrier layer to the prosthetic heart valve 100 in different or additional locations.

The barrier layer 124 can comprise a polymeric material. In some instances, the polymeric material can be relatively thromboresistant or have a thromboresistance that is greater than that of a fabric. In some instances, the polymeric material can be relatively thromboresistant or have a thromboresistance that is greater than that of the outer skirt 106. Further, the polymeric material can be relatively non-abrasive or smooth. In some instances, the polymeric material can be impermeable, such as impermeable to fluids. In other instances, the polymeric material can be partially impermeable, such as impermeable to cell migration and/or to prevent or limit neo-vascularization. In some instances, a partially impermeable polymeric material can include pores that are within the range of 0.5 µm to 20 µm (e.g., to prevent cell migration, to prevent or limit neo-vascularization, etc.). In some instances, a partially impermeable polymeric material can include pores that are within the range of 20 µm to 200 µm (e.g., to prevent or limit neo-vascularization, etc.). For example, in some instances, the polymeric material of the barrier layer 124 can comprise thermoplastic polyurethane (TPU), silicone, polyethylene terephthalate (PET), polytetrafluoroethylene (PTFE), expanded PTFE (ePTFE), and/or other similar materials. In some examples, the barrier layer 124 can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide, thermoplastic polyurethane (TPU), etc. In this way, the barrier layer 124 may prevent tissue ingrowth on the barrier layer 124 and/or onto the leaflets, prevent migration of cells from a location that is external to the barrier layer 124 towards a location that is radially inwards of the barrier layer 124, and/or improve laminar flow at the inflow end 108 of the prosthetic heart valve 100. In some examples, the barrier layer 124 can be a relatively thin layer or film that has a thickness of less than 15 µm, for example, between 0.5 µm and 15 µm, between 5 µm and 10 µm, etc.

While barrier layer 124 is shown as coupled to a balloon-expandable valve 100 in FIG. 3, any of the barrier layers described herein can be coupled to any of various valves that are balloon-expandable, self-expandable or mechanically expandable. Additional details on balloon expandable prosthetic valves can be found in U.S. Patent No. 9,393,110 and International Application PCT/US2022/25687, all of which are incorporated by reference herein. Additional details on a mechanically expandable prosthetic valve can be found in International Application PCT/US2021/052745, filed Sep. 30, 2021, and additional details on a self-expanding prosthetic valve can be found in U.S. Patent No. 8,652,202, all of which are incorporated by reference herein. Additional examples of prosthetic heart valves that can include the barrier layers disclosed herein can be found in U.S. Patent Nos. 11,013,600 and 11,096,781 and International Publication No. WO 2022/226147. Further, barrier layers described herein can be part of a prosthetic heart valve comprising one or more of the components described above for prosthetic valve 100, including frame 102, leaflets 112, and/or outer skirt 106.

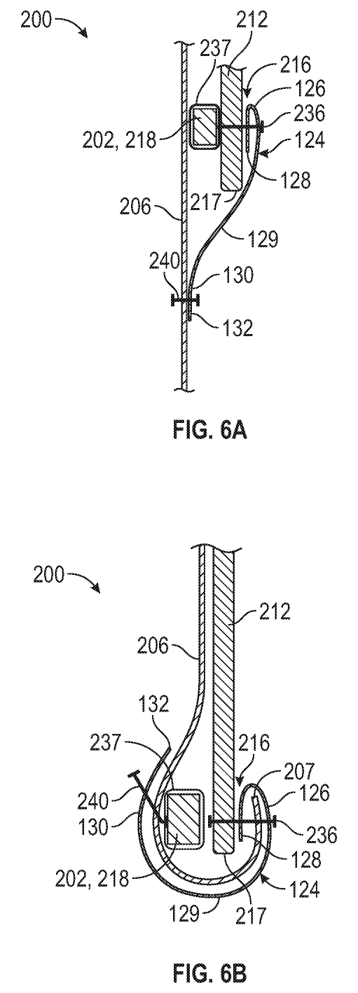

FIGS. 5–9 show various arrangements for the barrier layer 124 as included in an exemplary prosthetic heart valve 200. The prosthetic heart valve 200 is shown schematically and can comprise one or more of the components described above for prosthetic valve 100, as indicated by corresponding reference numbers. For example, similar to prosthetic heart valve 100, the prosthetic heart valve 200 can include a frame 202, an outer skirt 206, and a plurality of leaflets 212 disposed within the frame 202. Each leaflet 212 has a cusp edge portion 216 and an inflow edge 217 (see FIGS. 6A–6B). The prosthetic valve 200 can be balloon-expandable, self-expandable or mechanically expandable.

FIG. 5 illustrates the frame 202 in a flattened configuration and one leaflet 212 coupled to the frame 202 for purposes of illustration. It should be appreciated that the prosthetic heart valve 200 can include more than one leaflet 212 (e.g., three leaflets, etc.). FIG. 6A illustrates a cross-sectional view of the barrier layer 124 as arranged at an intermediate location of the frame 202 (e.g., between the inflow end 208 and the outflow end 210) (6A-6A of FIG. 5). FIG. 6B illustrates a cross-sectional view of the barrier layer 124 as arranged at the inflow end 208 of the frame 202 (6B-6B of FIG. 5). As shown, on the interior side of frame 202, the barrier layer 124 extends along and is coupled to the cusp edge portion 216 of the leaflet 212 as well as the outer skirt 206. Specifically, the first end portion 126 of the barrier layer 124 is coupled to the leaflet 212 and the second end portion 130 is coupled to the outer skirt 206. In some examples, the intermediate portion 129 and/or the second end portion 130 of the barrier layer 124 can extend around one or more struts 218 of the frame 202 (e.g., struts 218 at the inflow end 208 of the frame 202).

As shown in FIG. 6A, the first end portion 126 of the barrier layer 124 is in a folded configuration and coupled to the leaflet 212. Fasteners, such as in-and-out stitches 236, can pass through the first apertures 142 (FIG. 4) of the barrier layer 124, as well as through the leaflet 212 to secure the barrier layer 124 to the leaflet 212. In some examples, the first end portion 126 of the barrier layer 124 can be in an unfolded or non-folded configuration with the first edge 128 of the barrier layer 124 extending towards the outflow end 210 and adjacent to the leaflet 212. In some examples, fasteners, such as whip stitches 237, can extend through the in-and-out stitches 236 and around a strut 218 of the frame 202 to secure the leaflet 212 to the frame 202.

The second end portion 130 of the barrier layer 124 is coupled to a radially inner surface of the outer skirt 206 (e.g., with sutures). For example, sutures, such as in-and-out stitches 240, can pass through the second apertures 144 (FIG. 4) of the barrier layer 124, as well as through the outer skirt 206 to secure the barrier layer 124 to the leaflet 212. In some examples, a combination of suture stitches (e.g., whip stitches, in-and-out stitches, etc.) can be used to couple the barrier layer 124 to the leaflet 212 and outer skirt 206. In some examples, the second end portion 130 of the barrier layer 124 can be in a folded configuration, such that the second edge 132 is positioned axially between the outer skirt 206 and the intermediate portion 129 of the barrier layer 124 (e.g., with the second edge 132 extending towards the outflow end 210).

At the inflow end 208 of the frame 202 as shown in FIG. 6B, an inflow edge 207 of the outer skirt 206 can be wrapped around the frame 202 towards the interior of the frame 202. The inflow edge 207 of the outer skirt 206 can be positioned within the fold of the first end portion 126 of the barrier layer 124. Specifically, as shown in FIG. 6B, the intermediate portion 129 of the barrier layer 124 can be positioned radially inwards of the inflow edge 207 of the outer skirt 206 and the first edge 128 of the barrier layer 124 can be positioned radially outwards of the inflow edge 207 of the outer skirt 206. In this way, the inflow edge 207 of the outer skirt 206 can be surrounded by the barrier layer 124. In some examples, in-and-out stitches 236 can pass through the first apertures 142 of the barrier layer 124, through the outer skirt 206 (e.g., adjacent to the inflow edge 207), as well as through the leaflet 212 to secure the barrier layer 124 to the leaflet 212 and to secure the inflow edge 207 of the outer skirt 206 within the barrier layer 124. Additionally, at the second end portion 130 of the barrier layer 124, in-and-out stitches 240 can pass through the second apertures 144 of the barrier layer 124 and the outer skirt 206 to secure the barrier layer 124 to the outer skirt 206. In some examples, a combination of suture stitches (e.g., whip stitches, in-and-out stitches, etc.) can be used to couple the barrier layer 124 to the leaflet 212 and outer skirt 206 at the inflow end 208 of the frame 202.

In some examples, an outer skirt (e.g., any outer skirt described herein, outer skirt 106, outer skirt 206) of the prosthetic heart valve can be wrapped around the inflow end of the frame, such that the outer skirt extends from the exterior of the frame towards the interior of the frame. One example of a wrapped outer skirt is shown in FIG. 6B, although any outer skirt described herein can be wrapped around the inflow end of the frame. For example, as shown in FIG. 6B, the inflow edge 207 of the outer skirt 206 is wrapped around the inflow end 208 of the frame 202, such that the outer skirt 206 extends into the interior of the frame 202. In some examples, the inflow edge of the outer skirt can be disposed in the interior of the frame in a position that is radially between the frame and the barrier layer (e.g., barrier layer 124, barrier layer 324, barrier layer 424b, barrier layer 524b, etc.). In some examples, the inflow edge of the outer skirt can be disposed in the interior of the frame in a position that is radially inwardly of the barrier layer.

In some examples, wrapping the outer skirt around the inflow end of the frame can provide cushioning between the prosthetic heart valve and the native anatomy, which can prevent and/or reduce conduction disturbances as well as prevent and/or reduce tissue damage at areas of higher local stress. For example, cushioning can increase the contact area of the prosthetic heart valve with the host tissue such that the local stress is reduced, for example, at the inflow end of the frame. In some examples, a thicker outer skirt can be used to provide sufficient cushioning.

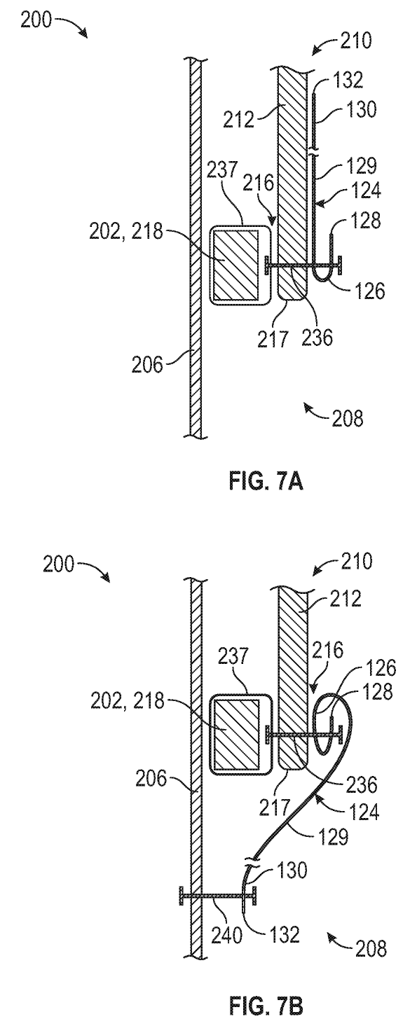

FIGS. 7A–B illustrates cross-sectional views of an example configuration of the barrier layer 124 as arranged at the intermediate location of the frame 202 (e.g., between the inflow end 208 and the outflow end 210) (similar to 6A-6A of FIG. 5). FIG. 7A shows the barrier layer 124 in a partially assembled state. When fully assembled (FIG. 7B), the intermediate portion 129 of the barrier layer 124 is positioned radially inwards of the in-and-out stitches 236. In this way, the stitches 236 can be covered by or enclosed within the barrier layer 124, which in some instances may further improve flow through the prosthetic valve 200.

As shown in FIG. 7A, the barrier layer 124 can initially be coupled to the leaflet 212 (e.g., with in-and-out stitches 236), such that the second end portion 130 of the barrier layer 124 extends towards the outflow end 210 adjacent to the leaflet 212. For example, the first end portion 126 can be in a folded configuration and secured to the leaflet 212 with sutures (e.g., in-and-out stitches 236). In this positioning (FIG. 7A), the first edge 128 and the second edge 132 of the barrier layer 124 both extend towards the outflow end 210. The barrier layer 124 can subsequently be folded towards the inflow end 208 and then coupled to a radially-inner surface of the outer skirt 206, as shown in FIG. 7B. In this way, the barrier layer 124 covers or encloses the stitches 236 within the barrier layer 124, which, in some instances, can reduce the likelihood of the stitches 236 disrupting fluid flow through the interior of the valve 200.

In some examples, the first end portion 126 of the barrier layer 124 can be in an unfolded configuration and also enclose sutures (e.g., in-and-out stitches 236) coupling the barrier layer 124 to the leaflet 212 within the barrier layer 124 (e.g., radially inwards of the intermediate portion 129 of the barrier layer 124). When fully assembled in the unfolded configuration, the first edge 128 and the second edge 132 of the barrier layer 124 extend towards the inflow end 208 of the valve 200 when the barrier layer 124 is coupled to the leaflet 212 and the outer skirt 206.

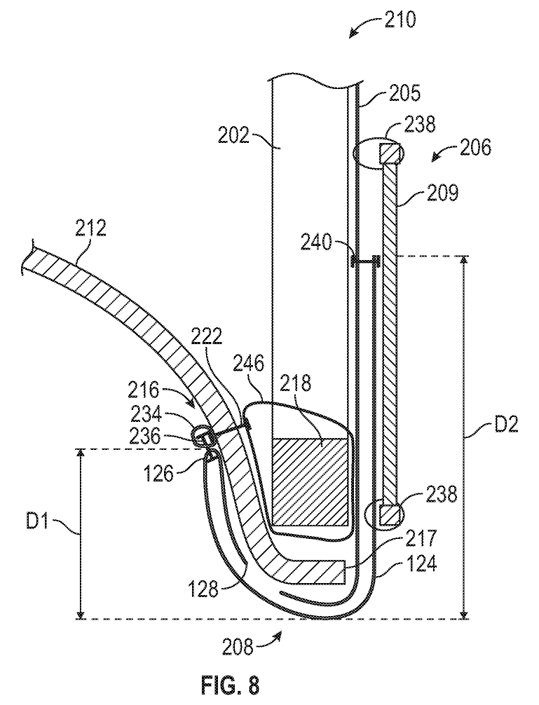

FIG. 8 illustrates an example configuration of the barrier layer 124 as included within the valve 200. As shown in FIG. 8, the outer skirt 206 includes multiple layers that are configured to promote endothelialization and/or tissue ingrowth between the native tissue and the outer skirt 206. Specifically, the outer skirt 206 includes an inner base layer 205 and an outer layer 209 that is coupled to the base layer 205. In this way, the base layer 205 can be configured as an anchoring layer to which the outer layer 209 is attached, for example, to maintain the position of the outer layer 209 relative to the frame 202. In some examples, the base layer 205 and the outer layer 209 can comprise different materials and/or textures. For example, the outer layer 209 can be a textured material that includes floating yarn portions that are texturized. Further details on such outer skirts can be found in U.S. Patent Application Publication 2019/0374337, International Publication WO 2021/202636, and U.S. Patent Application No. 63/366,599, filed Jun. 17, 2022, entitled “OUTER SKIRT FOR A PROSTHETIC HEART VALVE”, all of which are incorporated by reference herein.

In some examples, to encourage tissue ingrowth at the outer skirt 206 while preventing tissue ingrowth from occurring on the leaflets 212 and/or within the frame 202, the barrier layer 124 can be at least partially disposed between the base layer 205 and the outer layer 209 of the outer skirt 206. As described above, the barrier layer 124 can also be configured to enclose or secure the free portions of the leaflets 212 to create a smoother transition at the inflow edge 217 of the leaflet 212. FIG. 8 shows the barrier layer 124 coupled to the outer skirt 206 and the leaflet 212, for example, at the inflow end 208 of the frame 202. As shown, the barrier layer 124 is coupled to the cusp edge portion 216 of the leaflet 212 and disposed around the inflow edge 217 of the leaflet 212. In this way, the barrier layer 124 can enclose or surround the inflow edge 217 of the leaflet 212, such that the inflow edge 217 is disposed between the barrier layer 124 and the frame 202.

In some examples, as illustrated in FIG. 8, the first end portion 126 is positioned radially inwards of the frame 202 (e.g., within the frame 202) and is coupled, for example, to the cusp edge portion 216 of the leaflet 212 (e.g., similar to the configuration shown in FIG. 3). In some examples, the distance D1 between the inflow end 208 and the first end portion 126 of the barrier layer 124 that is positioned closest to the outflow end 210 is within the range of 0.5 mm to 3 mm. While the first end portion 126 is illustrated in a folded configuration in FIG. 8, in some examples, the first end portion 126 can be in an unfolded configuration, such that the first edge 128 extends axially towards the outflow end 210 of the valve.

With continued reference to FIG. 8, the second end portion 130 is positioned radially outside of the frame 202 and coupled to the outer skirt 206 in a position that is radially between the base layer 205 and the outer layer 209 of the outer skirt 206. For example, a portion of the base layer 205 (e.g., an inflow end portion) can be wrapped around the inflow end 208 of the frame 202 and enclosed or surrounded by the intermediate portion 129 of the barrier layer 124. The second end portion 130 of the barrier layer 124 can extend in an axial direction towards the outflow end 210 of the valve 200 at an intermediate position of the outer skirt 206 (e.g., between inflow and outflow ends of the skirt 206). In some examples, the distance D2 between the inflow end 208 of the valve 200 and the second edge 132 of the barrier layer 124 is within the range of 3 mm to 10 mm.

As shown in FIG. 8, fasteners or sutures, such as sutures comprising whip stitches and/or in-and-out stitches, can be used to secure the first end portion 126 of the barrier layer 124 radially inward of the frame 202 (e.g., to the leaflet 212). Specifically, in some examples, in-and-out stitches 236 can extend through the barrier layer 124. First whip stitches 234 can pass through the in-and-out stitches 236 at the barrier layer 124 and the in-and-out stitches 222 of the leaflet 212, as shown in FIG. 8, to secure the barrier layer 124 to the leaflet 212. In some examples, second whip stitches 246 can extend through the in-and-out stitches 222 of the leaflet 212 and around the frame 202 and/or the base layer 205 of the outer skirt 206 to secure the leaflet 212 to the frame 202. Fasteners or sutures, such as sutures comprising whip stitches and/or in-and-out stitches, can be used to secure the second end portion 130 of the barrier layer 124 radially outward of the frame 202 (e.g., to the outer skirt 206). Specifically, in some examples, whip stitches 238 can extend through the outer layer 209 of the outer skirt 206 and the barrier layer 124 to secure the outer layer 209 and the barrier layer 124 together. In-and-out stitches 240 can extend through the barrier layer 124 and the base layer 205 to secure the barrier layer 124 to the base layer 205. Whip stitches 238 can also extend through the outer layer 209 of the outer skirt 206 and the base layer 205 of the outer skirt 206. Other configurations of fasteners or sutures (e.g., whip stitches, in-and-out stitches, other stitches, etc.) can be used to couple the barrier layer 124 to the inflow end 108 of the prosthetic heart valve 100.

In some examples, the barrier layer 124 can include additional apertures (e.g., in addition to first and second apertures 142, 144, etc.) to accommodate other fasteners, such as those used to couple the outer layer 209 of the outer skirt 206 to barrier layer 124 (e.g., whip stitches 238, etc.).

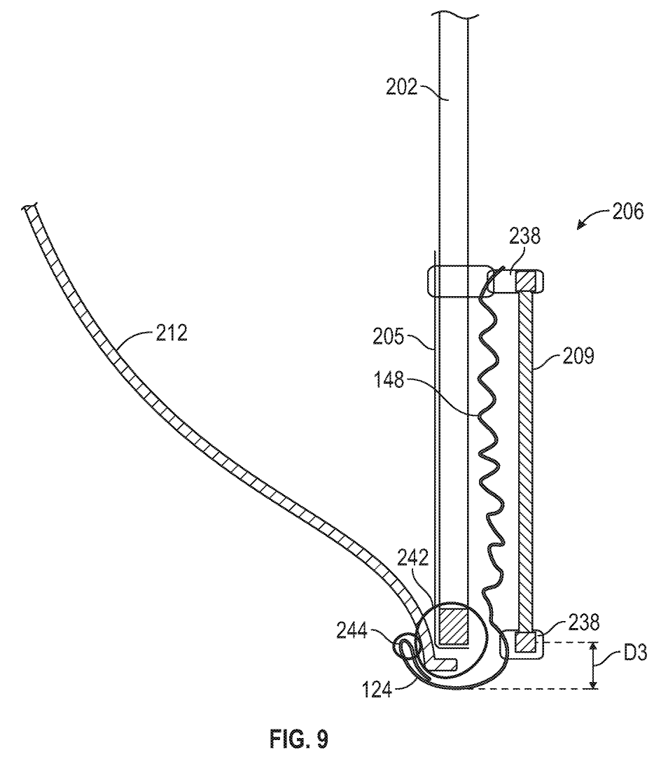

While the base layer 205 is illustrated radially outwards of the frame 202 in FIG. 8, in some examples, the base layer 205 can be positioned radially inwards of or within the frame 202 (e.g., as an inner skirt), as shown in FIG. 9. In some examples, the barrier layer 124 can serve as an anchoring layer for the outer layer 209 of the outer skirt 206 (e.g., to maintain the position of the outer layer 209 relative to the frame 202, etc.) in addition to and/or in lieu of the base layer 205. For example, as shown in FIG. 9, fasteners, such as whip stitches 238, can be used to secure the outer layer 209 of the outer skirt 206 to the barrier layer 124 and/or to the base layer 205. In some examples, a prosthetic heart valve can include the outer skirt 206 with the base layer 205 positioned radially outwards of the frame 202 and include an inner skirt disposed within the frame.

In some instances, as shown in FIG. 9, multiple whip stitches can be used to secure the leaflet 212 to the frame 202 and to secure the barrier layer 124 to the leaflet 212. Specifically, first whip stitches 242 can extend through the leaflet 212 and around the frame 202 (e.g., around a strut 218 of the frame 202). Second whip stitches 244 can extend through the barrier layer 124 (e.g., through first apertures 142) and through the first whip stitches 242. It should be appreciated that other configurations of sutures (e.g., in-and-out stitches, whip stitches, and/or other stitches) can be used to secure the leaflet 212, the frame 202 and/or the barrier layer 124 together.

In some examples, as illustrated in FIG. 9, it can be beneficial to create physical space, separation, or a gap between the outer layer 209 of the outer skirt 206 (e.g., a portion of the outer skirt 206 configured to promote endothelialization and/or tissue ingrowth between the native tissue and the outer skirt 206) and the leaflet 212. This space can be beneficial in both the radial direction and/or the axial direction and can be achieved by the barrier layers described herein (e.g., barrier layer 124). In some examples, this space can be represented in the axial direction by distance D3. In some examples, the distance D3 between the inflow end 208 and the end of the outer layer 209 of the outer skirt 206 that is positioned closest to the inflow end 208 is within the range of 0.5 mm to 2 mm.

In some examples, the barrier layer 124 can comprise a non-stretchable material or relatively non-stretchable material (e.g., less stretchable than the outer skirt 206). Non-limiting examples of non-stretchable or inelastic materials include polyurethane (TPU), silicone, and the like. To accommodate the expansion of the valve 200 when the valve 200 is radially expanded to its deployed configuration, the barrier layer 124 can include one or more folds 148 that provide additional material or slack to permit expansion of the valve 200. In some examples, as shown in FIG. 9, the barrier layer 124 can comprise a stretchable material and include one or more folds 148. Non-limiting examples of stretchable or elastic materials include polyethylene terephthalate (PET), polytetrafluoroethylene (PTFE), expanded PTFE (ePTFE), and the like.

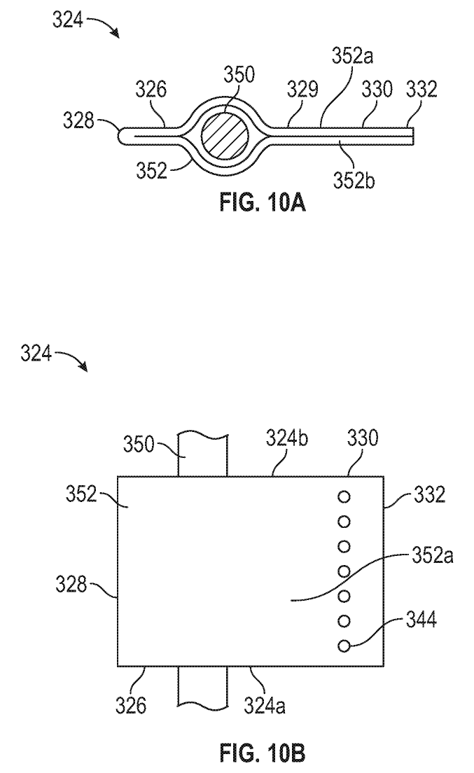

FIGS. 10A-10B illustrate a barrier layer 324, according to an example. The barrier layer 324 can be a part of any prosthetic heart valve described herein (e.g., prosthetic heart valve 100, prosthetic heart valve 200, etc.). In some examples, the barrier layer 324 can be used instead of barrier layer 124. The barrier layer 324 includes a suture 350 and a polymeric layer 352 disposed around the suture 350, such that the suture 350 and the polymeric layer 352 are coupled together. In some examples, the suture 350 and the polymeric layer 352 can be coupled together by a lamination process. In some examples, the suture 350 can be secured within the polymeric layer 352, such that the suture 350 is circumferentially enclosed or surrounded by the polymeric layer 352 along a length of the suture 350. FIG. 10A shows space between the suture 350 and the polymeric layer 352 for purposes of illustration, however, it should be appreciated that in some instances, the polymeric layer 352 contacts at least a portion of the outer surface of the suture 350.

Similar to barrier layer 124, barrier layer 324 includes a first end portion 326 having a first edge 328 and a second end portion 330 having a second edge 332. The barrier layer 324 can also include an intermediate portion 329 between the first end portion 326 and the second end portion 330. As shown in FIG. 10B, the barrier layer 324 can include a plurality of apertures 344 positioned along the length of the second end portion 330, for example, from a first side 324a to a second side 324b of the barrier layer 324. While one row of apertures 344 is shown, in some examples, the barrier layer 324 can include multiple rows of apertures 344. It should be appreciated that dimensions of the barrier layer 324 may not be to scale. For example, a width of the barrier layer 324 (e.g., from the first side 324a to the second side 324b) can be long enough to extend along the entire cusp edge portion of a leaflet (e.g., leaflet 112, leaflet 212, etc.) and/or a majority of the cusp edge portion.

The suture 350 can be positioned between an upper portion 352a of the polymeric layer 352 and a lower portion 352b of the polymeric layer 352. In some examples, the polymeric layer 352 can be a single layer that is folded around the suture 350. For example, as shown in FIG. 10A, the polymeric layer 352 can have a fold at one of the ends (e.g., at the first end portion 326) to define the upper portion 352a and the lower portion 352b. In some examples, the polymeric layer 352 can comprise multiple layers. For example, rather than being folded at one of the ends, a first polymeric layer can define the upper portion 352a and a separate, second polymeric layer can define the lower portion 352b.

The suture 350 can disposed within the first end portion 326 of the barrier layer 324 and extend through the polymeric layer 352. For example, in some instances, as depicted, the suture 350 can extend beyond the first side 324a and the second side 324b of the polymeric layer 352. In some examples, the suture 350 can be fully enclosed within the polymeric layer 352 at the first side 324a and the second side 324b, such that the suture 350 does not extend beyond the first side 324a and the second side 324b of the polymeric layer 352.

In some examples, the suture 350 can comprise a relatively larger suture than those described above as fasteners for coupling various components together. For example, the suture 350 can comprise a 3-0 suture. The polymeric layer 352 can comprise a polymeric material. In some instances, the polymeric material can be relatively thromboresistant or have a thromboresistance that is greater than that of a fabric. In some instances, the polymeric material can be relatively thromboresistant or have a thromboresistance that is greater than that of the outer skirts described herein (e.g., outer skirt 106, 206, etc.). Further, the polymeric material can be relatively non-abrasive or smooth. In some instances, the polymeric material can be impermeable, such as impermeable to fluids. In other instances, the polymeric material can be partially impermeable, such as impermeable to cell migration and/or to prevent or limit neo-vascularization. In some instances, a partially impermeable polymeric material can include pores that are within the range of 0.5 µm to 20 µm (e.g., to prevent cell migration, to prevent or limit neo-vascularization, etc.). In some instances, a partially impermeable polymeric material can include pores that are within the range of 20 µm to 200 µm (e.g., to prevent or limit neo-vascularization, etc.). For example, in some instances, the polymeric material of the polymeric layer 352 can comprise thermoplastic polyurethane (TPU), silicone, polyethylene terephthalate (PET), polytetrafluoroethylene (PTFE), expanded PTFE (ePTFE), and/or other similar materials. In some examples, the barrier layer 324 can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide, thermoplastic polyurethane (TPU), etc. In this way, the barrier layer 324 may prevent tissue ingrowth on the polymeric layer 352 and/or onto the leaflets, prevent migration of cells from a location that is external to the barrier layer 324 towards a location that is radially inwards of the barrier layer 324, and/or improve laminar flow at the inflow end of a prosthetic heart valve (e.g., prosthetic heart valve 100, prosthetic heart valve 200, etc.). In some examples, the polymeric layer 352 can be a relatively thin layer or film that has a thickness of less than 15 µm, for example, between 0.5 µm and 15 µm, between 5 µm and 10 µm, etc.

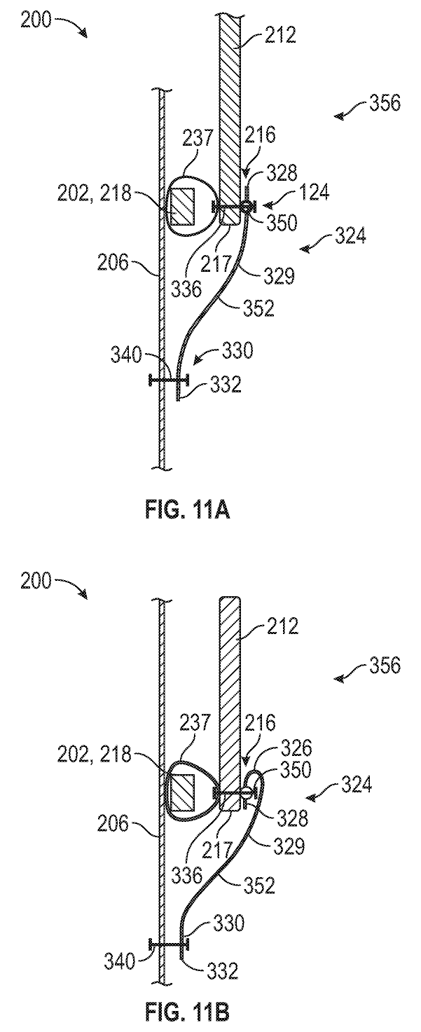

FIGS. 11A–11B illustrate examples of the barrier layer 324 as included within the prosthetic heart valve 200. Specifically, FIG. 11A illustrates a cross-sectional view of the barrier layer 324 as arranged at an intermediate location of the frame 202 (e.g., between the inflow end 208 and the outflow end 210) (similar to 6A-6A of FIG. 5), according to one example. As shown in FIG. 11A, the first end portion 326 of the barrier layer 324 is coupled to the leaflet 212. Specifically, the suture 350 is positioned adjacent to the cusp edge portion 216 of the leaflet 212 and the first edge 328 of the barrier layer 324 extends towards the outflow end 210. Fasteners 336, such as in-and-out stitches and/or whip stitches, can pass through the suture 350, as well as through the leaflet 212 to secure the barrier layer 324 to the leaflet 212. In some examples, the fasteners 336 can extend through the polymeric layer 352 and around the suture 350. In this way, the suture 350 can provide additional strain relief at the cusp edge portion 216 of the leaflet 212. Fasteners, such as whip stitches 354, can extend through the fasteners 336 and around the frame 202 to secure the leaflet 212 and the barrier layer 324 to the frame 202.

In some examples, the barrier layer 324 and the leaflets 212 can be collectively referred to as a leaflet construct 356 (FIGS. 11A–11B). Specifically, the leaflet construct 356 can include an equal number of barrier layers 324 and leaflets 212 (e.g., one barrier layer 324 per leaflet 212, etc.) and be formed separately from the frame 202 and subsequently attached thereto with fasteners (e.g., whip stitches 354). In some examples, the leaflet construct 356 can comprise three leaflets 212 and three barrier layers 324. In some examples, a leaflet construct can comprise any barrier layer described herein (e.g., barrier layer 124 and/or barrier layer 324) and any leaflet described herein (e.g., leaflet 112 and/or leaflet 212).

Similar to barrier layer 124, the second end portion 330 of the barrier layer 324 can be coupled to a radially inner surface of the outer skirt 206 (e.g., with fasteners such as sutures). For example, fasteners, such as in-and-out stitches 340, can pass through the apertures 344 of the barrier layer 324, as well as through the outer skirt 206 to secure the barrier layer 324 to the leaflet 212. In some examples, a combination of suture stitches (e.g., whip stitches, in-and-out stitches, etc.) can be used to couple the barrier layer 324 to the leaflet 212 and outer skirt 206. In some examples, the second end portion 330 of the barrier layer 324 can be in a folded configuration, such that the second edge 332 is positioned axially between the outer skirt 206 and the intermediate portion 329 of the barrier layer 324 (e.g., with the second edge 332 extending towards the outflow end 210).

FIG. 11B illustrates a cross-sectional view of the barrier layer 324 as arranged at an intermediate location of the frame 202 (e.g., between the inflow end 208 and the outflow end 210) (similar to 6A-6A of FIG. 5), according to one example. FIG. 11B is similar to FIG. 11A, except that the first edge 328 of the barrier layer 324 extends towards the inflow end 208, rather than towards the outflow end 210. In this way, the barrier layer 324 can be folded, for example, similarly to FIGS. 7A–7B, to cover and/or enclose the suture 350 and the fasteners 336 within the barrier layer 324. The intermediate portion 329 of the barrier layer 324 is positioned radially outwards of the suture 350 and fasteners 336, which, in some instances, can reduce the likelihood of the suture 350 and/or the fasteners 336 disrupting fluid flow through the interior of the valve 200.

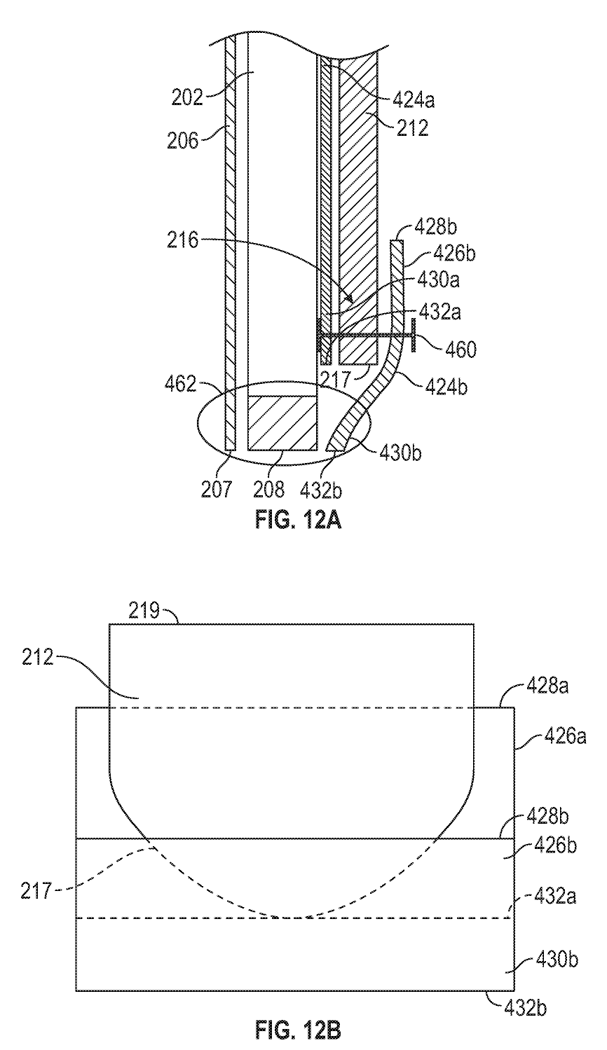

In some examples, any prosthetic heart valve described herein can include multiple barrier layers. FIGS. 12A–12B illustrate a first barrier layer 424a and a second barrier layer 424b as included within the prosthetic heart valve 200, according to one example. FIG. 12A illustrates a cross-sectional view of the barrier layers 424a, 424b as arranged at the inflow end 208 of the frame 202 (similar to 6B-6B of FIG. 5). Similar to barrier layer 124, the first barrier layer 424a includes a first end portion 426a having a first edge 428a and a second end portion 430a having a second edge 432a and the second barrier layer 424b includes a first end portion 426b having a first edge 428b and a second end portion 430b having a second edge 432b.

The barrier layers 424a, 424b can comprise a polymeric material. In some instances, the polymeric material can be relatively thromboresistant or have a thromboresistance that is greater than that of a fabric. In some instances, the polymeric material can be relatively thromboresistant or have a thromboresistance that is greater than that of the outer skirt 106. Further, the polymeric material can be relatively non-abrasive or smooth. In some instances, the polymeric material can be impermeable, such as impermeable to fluids. In other instances, the polymeric material can be partially impermeable, such as impermeable to cell migration and/or to prevent or limit neo-vascularization. In some instances, a partially impermeable polymeric material can include pores that are within the range of 0.5 µm to 20 µm (e.g., to prevent cell migration, to prevent or limit neo-vascularization, etc.). In some instances, a partially impermeable polymeric material can include pores that are within the range of 20 µm to 200 µm (e.g., to prevent or limit neo-vascularization, etc.). For example, in some instances, the polymeric material of the barrier layers 424a, 424b can comprise thermoplastic polyurethane (TPU), silicone, polyethylene terephthalate (PET), polytetrafluoroethylene (PTFE), expanded PTFE (ePTFE), and/or other similar materials. In some examples, the barrier layers 424a, 424b can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide, thermoplastic polyurethane (TPU), etc. In this way, the barrier layers 424a, 424b may prevent tissue ingrowth on the barrier layers 424a, 424b and/or onto the leaflets, prevent migration of cells from a location that is external to the barrier layers 424a, 424b toward a location that is radially inwards of the barrier layers 424a, 424b, and/or improve laminar flow at the inflow end of a prosthetic heart valve (e.g., prosthetic heart valve 100, prosthetic heart valve 200, etc.). In some examples, the barrier layers 424a, 424b can be a relatively thin layer or film that has a thickness of less than 15 µm, for example, between 0.5 µm and 15 µm, between 5 µm and 10 µm, etc.

As shown in FIG. 12A, the first barrier layer 424a is disposed in an interior of the frame 202. Specifically, the first barrier layer 424a is positioned axially between the frame 202 and the leaflet 212. In this way, the first barrier layer 424a can be configured to prevent cell growth (e.g., smooth muscle cells, etc.) from migrating from an exterior of the frame 202 towards an interior of the frame 202 and the leaflets 212. The second end portion 430a of the first barrier layer 424a can be coupled to struts 218 of the frame 202 with fasteners, for example, adjacent to the inflow end 208 of the frame 202. In some examples, the second end portion 430a is spaced apart from the inflow end 208 of the frame 202.

The first end portion 426a can also be coupled to struts 218 of the frame 202 with fasteners, for example, adjacent to the outflow end 210 of the frame 202. In some examples, the first barrier layer 424a can be positioned such that the first end portion 426a is spaced apart from the coaptation edge 219 of the leaflets 212. For example, as shown in FIG. 12B, the first edge 428a can be positioned closer to the inflow end 208 than the coaptation edge 219. In some examples, the first edge 428a can be axially aligned with an outflow edge of the outer skirt 206. In FIG. 12B, only one leaflet 212 is shown for purposes of illustration. While the first barrier layer 424a and the second barrier layer 424b are shown as extending to the sides of the leaflet 212 by a distance, each of the first barrier layer 424a and the second barrier layer 424b fully extends around the interior of the frame 202 in the circumferential direction.

The cusp edge portion 216 of the leaflet 212 is coupled to the first barrier layer 424a and the second barrier layer 424b with fasteners 460. In some examples, the leaflet 212 is coupled to the barrier layers 424a, 424b with fasteners 460, such that the cusp edge 217 of the leaflet 212 is positioned at a location that is axially spaced apart from the inflow end 208 of the frame 202. As shown in FIG. 12A, the fasteners 460 can extend through the second end portion 430a of the first barrier layer 424a adjacent to the second edge 432a, through the cusp edge portion 216 of the leaflet 212 adjacent to the edge 217, and through the second barrier layer 424b at a location that is spaced apart from the first edge 428b. In this way, the first edge 428b of the second barrier layer 424b can be considered a free edge. As such, during coaptation of the leaflets 212, the free edge 428b of the second barrier layer 424b is able to move radially inwards and outwards with the leaflets 212.

The second barrier layer 424b is disposed in the interior of the frame 202 and is positioned radially inwardly of the cusp edge 217 of the leaflet 212. In this way, the first barrier layer 424a and the second barrier layer 424b from a pocket in which the cusp edge portion 216 of the leaflet 212 is received. The second end portion 430b of the second barrier layer 424b is coupled to the frame 202 with fasteners 462, adjacent to the inflow end 208 of the frame 202. In some examples, as depicted, the fasteners 462 can be used to couple both of the second barrier layer 424b and the outer skirt 206 to the inflow end 208 of the frame 202.

As shown in FIG. 12A, the second edge 432a of the first barrier layer 424a can overlap in the axial direction with the first edge 428b of the second barrier layer 424b. For example, the second edge 432a of the first barrier layer 424a is positioned closer to the inflow end 208 of the frame 202 than the first edge 428b of the second barrier layer 424b.

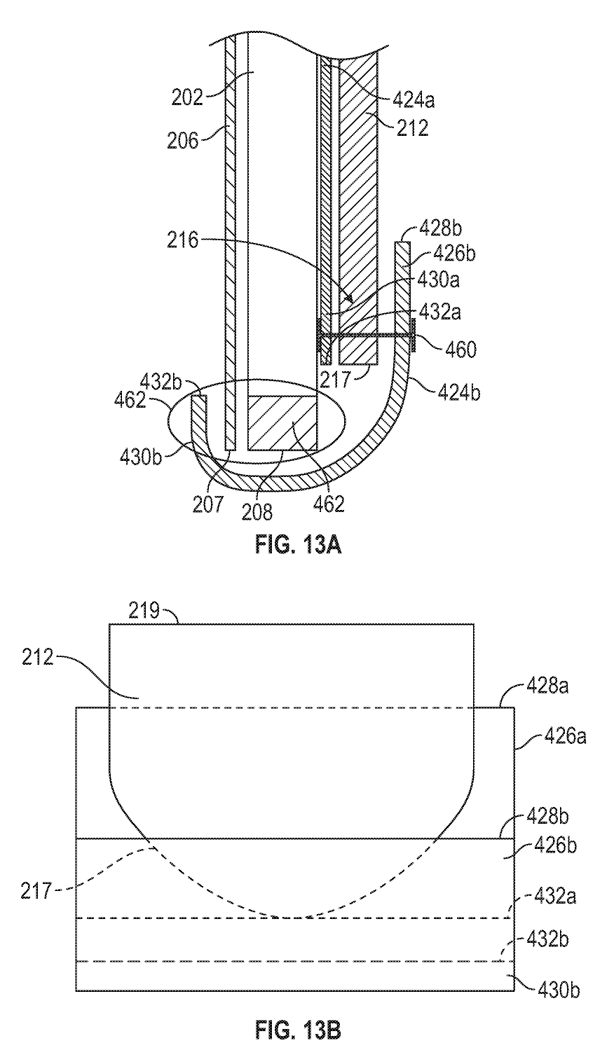

In some examples, the second barrier layer 424b can wrap or extend around the inflow end 208 of the frame 202, as shown in FIGS. 13A–13B. For example, the second end portion 430b of the second barrier layer 424b is disposed radially outwardly of the frame 202 and the first end portion 426b of the second barrier layer 424b is disposed radially inwardly of the frame 202. In some examples, the inflow edge 207 of the outer skirt 206 can be positioned radially between the second end portion 430b of the second barrier layer 424b and the frame 202. In this way, the second barrier layer 424b can surround or enclose the inflow edge 207 of the outer skirt 206 and the inflow end 208 of the frame 202, which in some instances may further improve flow through the prosthetic valve 200.

As shown in FIGS. 12B and 13B, the edges of the first barrier layer 424a and the second barrier layer 424b (e.g., edges 428a, 432a, 428b, 432b, respectively) are generally straight and extend around the interior of the frame 202 in the circumferential direction. For example, as shown, the first edge 428b of the second barrier layer 424b extends across the cusp edge portion 216 of the leaflet 212 and covers a portion of the cusp edge 217 (e.g., the portion of the cusp edge 217 adjacent to the inflow end 208). As described above, covering at least a portion of the cusp edge 217 of the leaflet 212 can improve laminar flow through the prosthetic heart valve 200 by creating a smoother transition at the cusp edge 217 of the leaflet 212.

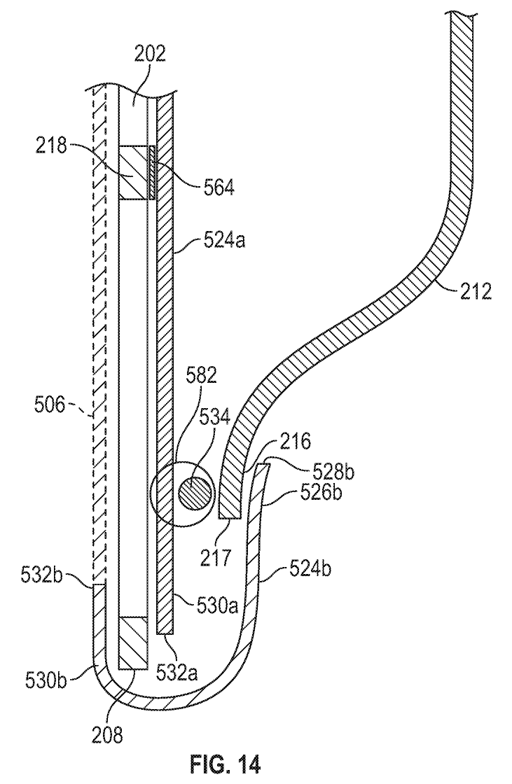

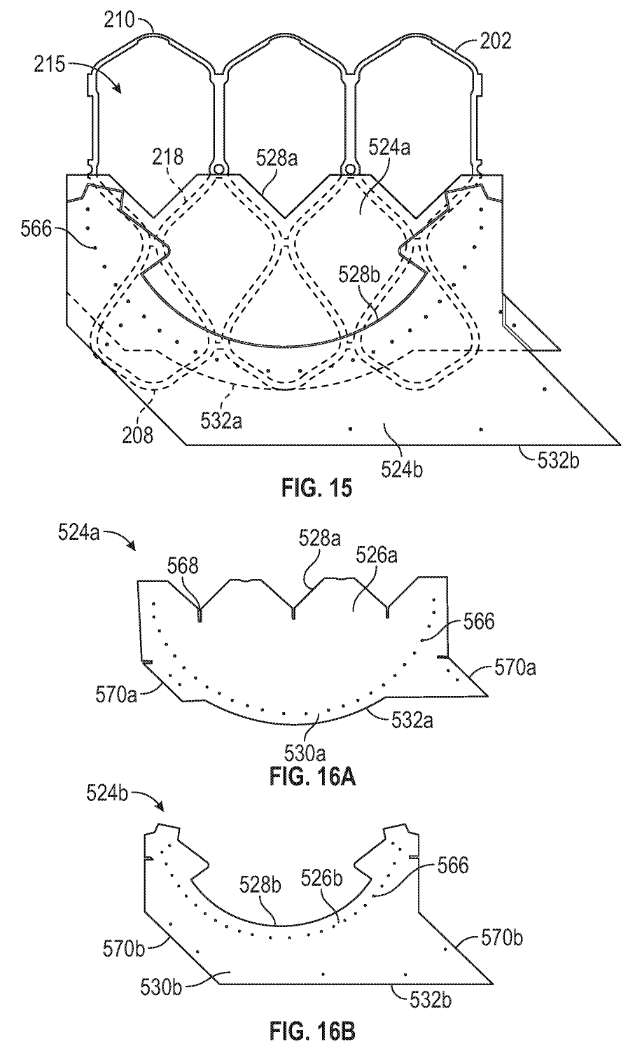

In some examples, the edges of the first and second barrier layers that are adjacent to the cusp edge 217 of the leaflet 212 (e.g., the second edge 432a of the first barrier layer 424a and the first edge 428b of the second barrier layer 424b) can comprise curved or scalloped edges that follow the shape of the cusp edge 217 of the leaflet 212. For example, FIGS. 14–16B illustrate a first barrier layer 524a and a second barrier layer 524b that have curved or scalloped edges as included within the prosthetic heart valve 200, according to one example. FIG. 14 illustrates a cross-sectional view of the barrier layers 524a, 524b as arranged at the inflow end 208 of the frame 202 (similar to 6B-6B of FIG. 5). Similar to barrier layers 424a and 424b, the first barrier layer 524a includes a first end portion 526a having a first edge 528a (FIG. 15) and a second end portion 530a having a second edge 532a and the second barrier layer 524b includes a first end portion 526b having a first edge 528b and a second end portion 530b having a second edge 532b.

The barrier layers 524a, 524b can comprise a polymeric material. In some instances, the polymeric material can be relatively thromboresistant or have a thromboresistance that is greater than that of a fabric. In some instances, the polymeric material can be relatively thromboresistant or have a thromboresistance that is greater than that of the outer skirt 106. Further, the polymeric material can be relatively non-abrasive or smooth. In some instances, the polymeric material can be impermeable, such as impermeable to fluids. In other instances, the polymeric material can be partially impermeable, such as impermeable to cell migration and/or to prevent or limit neo-vascularization. In some instances, a partially impermeable polymeric material can include pores that are within the range of 0.5 µm to 20 µm (e.g., to prevent cell migration, to prevent or limit neo-vascularization, etc.). In some instances, a partially impermeable polymeric material can include pores that are within the range of 20 µm to 200 µm (e.g., to prevent or limit neo-vascularization, etc.). For example, in some instances, the polymeric material of the barrier layers 524a, 524b can comprise thermoplastic polyurethane (TPU), silicone, polyethylene terephthalate (PET), polytetrafluoroethylene (PTFE), expanded PTFE (ePTFE), and/or other similar materials. In some examples, the barrier layers 524a, 524bcan comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide, thermoplastic polyurethane (TPU), etc. In this way, the barrier layers 524a, 524b may prevent tissue ingrowth on the barrier layers 524a, 524b and/or onto the leaflets, prevent migration of cells from a location that is external to the barrier layers 524a, 524b toward a location that is radially inwards of the barrier layers 524a, 524b, and/or improve laminar flow at the inflow end of a prosthetic heart valve (e.g., prosthetic heart valve 100, prosthetic heart valve 200, etc.). In some examples, the barrier layers 524a, 524b can be a relatively thin layer or film that has a thickness of less than 15 µm, for example, between 0.5 µm and 15 µm, between 5 µm and 10 µm, etc.

As shown in FIG. 14, the first barrier layer 524a is disposed in an interior of the frame 202. Specifically, the first barrier layer 524a is positioned axially between the frame 202 and the leaflet 212. In this way, the first barrier layer 524a can be configured to prevent cell growth (e.g., smooth muscle cells, etc.) from migrating from an exterior of the frame 202 towards an interior of the frame 202 and the leaflets 212. In some examples, as depicted, at least a portion of the second edge 532a can extend beyond the cusp edge 217 of the leaflet 212 towards the inflow end 208 of the frame 202. For example, the cusp edge 217 of the leaflet 212 can be spaced apart from the inflow end 208 of the frame 208 and the second edge 532a can be positioned closer to the inflow end 208 than the cusp edge 217. The first barrier layer 524a can be coupled to the frame 202 as described in more detail below.

The second barrier layer 524b is disposed in the interior of the frame 202 and is positioned radially inwardly of the cusp edge 217 of the leaflet 212. In this way, the first barrier layer 524a and the second barrier layer 524b from a pocket in which the cusp edge portion 216 of the leaflet 212 is received. In some examples, as depicted, the second end portion 530b of the second barrier layer 524b wraps around the inflow end 208 of the frame 202. Optionally, an outer skirt 506 is integrated with the second barrier layer 524b and extends from the second end portion 530b of the second barrier layer 524b (e.g., extends from the second barrier layer 524b adjacent to the inflow end 208 of the frame 202 towards the outflow end 210 of the frame 202). The outer skirt 506 can include any material described herein for outer skirt 106 and outer skirt 206. The second barrier layer 524b can be coupled to the frame 202 as described in more detail below.

In some examples, the second barrier layer 524b can wrap or extend around the inflow end 208 of the frame 202, as shown in FIG. 14. For example, the second end portion 530b of the second barrier layer 524b is disposed radially outwardly of the frame 202 and the first end portion 526b of the second barrier layer 524b is disposed radially inwardly of the frame 202. In some examples, an outer skirt 506 can optionally be integrated with the second barrier layer 524b, such that the outer skirt 506 extends from the second end portion 530b of the second barrier layer 524b towards the outflow end 210 of the frame 202.

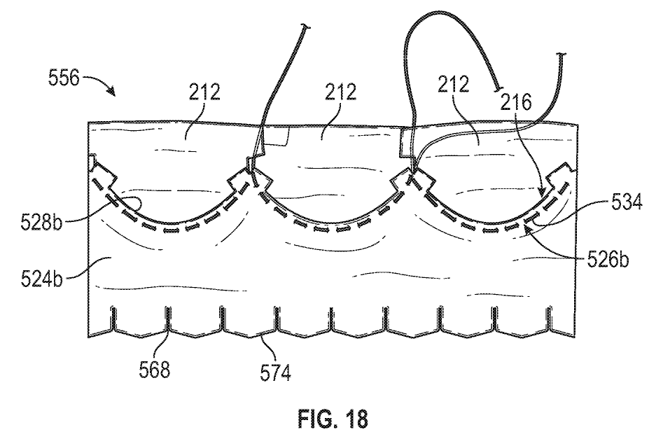

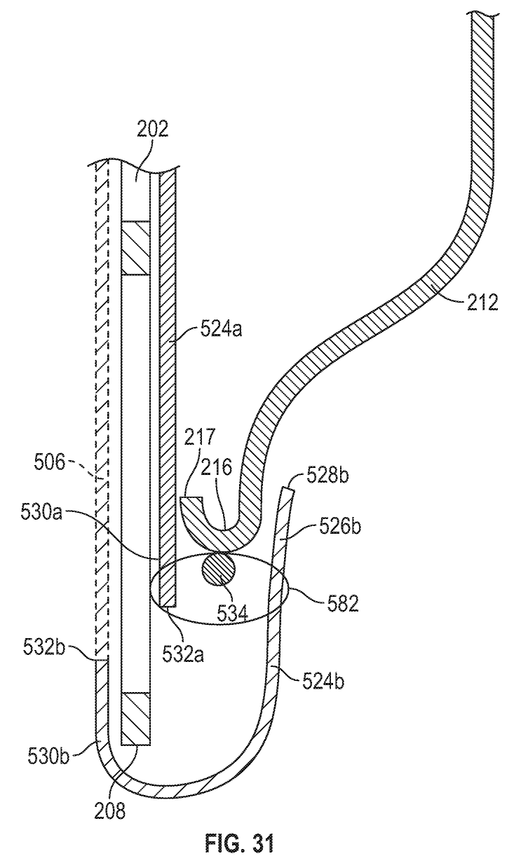

In some examples, a pledget 534 can be coupled to the cusp edge portion 216 of the leaflet 212. For example, the pledget 534 can extend through the cusp edge portion 216 of the leaflet 212 in an in-and-out pattern. The pledget 534 can comprise a thin strip of fabric or a suture (e.g., a 4-0 suture, a 5-0 suture, etc.). In some examples, the pledget 534 can comprise multiple strips of fabric or sutures. In some examples, the pledget 534 can comprise a single strip of fabric or a single suture. The pledget 534 can reinforce the leaflet 212 and provide a location for other components to be coupled to the leaflet 212. The second barrier layer 524b can be coupled to the leaflet 212 via the pledget 534. In some examples, the second barrier layer 524b can be coupled to the pledget 534 with fasteners (e.g., sutures, etc.) that extend around or through the pledget 534 and around or through the first end portion 526b of the second barrier layer 524b (FIG. 31). In some examples, the pledget 534 can extend through both the cusp edge portion 216 of the leaflet 212 and the first end portion 526b of the second barrier layer 524b in an in-and-out pattern (FIG. 18). In some examples, the second barrier layer 524b, the pledget 534, and the leaflets 212 can be collectively referred to as a leaflet construct 556 (FIG. 18).

In some examples, the first barrier layer 524a is coupled to the leaflet 212 and/or pledget 534 with fasteners 582, such as sutures. As described in more detail below, the fasteners 582 can extend around and/or through the second end portion 530a of the first barrier layer 524a, the pledget 534, and/or the leaflet 212. In some examples, the first barrier layer 524a is coupled to the leaflet 212 with the pledget 534, as described in more detail below.

The first barrier layer 524a can be coupled to the frame 202. In some examples, the first barrier layer 524a can be coupled to the frame 202 in a suture-less manner, without the use of sutures. As shown in FIG. 14, the first barrier layer 524a can be fused to the frame 202, for example, with adhesive 564. In some examples, the adhesive 564 can be positioned radially between one or more struts 218 of the frame 202 and the first barrier layer 524a. In some examples, as described in more detail below, the first barrier layer 524a can be coupled to the frame 202 with fasteners, such as sutures, in addition to or in lieu of the adhesive 564.

As shown in FIG. 14, the second edge 532a of the first barrier layer 524a can overlap in the axial direction with the first edge 528b of the second barrier layer 524b. For example, the second edge 532a of the first barrier layer 524a is positioned closer to the inflow end 208 of the frame 202 than the first edge 528b of the second barrier layer 524b.

The edges of the first and second barrier layers 524a, 524b that are adjacent to the cusp edge 217 of the leaflet 212 (e.g., the second edge 532a of the first barrier layer 524a and the first edge 528b of the second barrier layer 524b) can comprise curved or scalloped edges that generally follow the shape of the cusp edge 217 of the leaflet 212, as shown in FIGS. 15–16B. FIG. 15 depicts a portion of the frame 202 and segments of the first barrier layer 524a and the second barrier layer 524b without the optional outer skirt 506. The leaflet 212 is omitted from FIG. 15 for purposes of illustration. FIG. 16A illustrates the first barrier layer 524a and FIG. 16B illustrates the second barrier layer 524b without the optional outer skirt 506.

As shown, the second edge 532a of the first barrier layer 524a and the first edge 528b of the second barrier layer 524b are curved or scalloped shaped to correspond with the curved cusp edge portion 216 of the leaflet 212. For example, the second end portion 530a of the first barrier layer 524a and the first end portion 526b of the second barrier layer 524b can include holes 566 that follow the curved shape of the cusp edge portion 216 of the leaflet 212. As described in more detail below, fasteners (e.g., sutures) can pass through the holes 566 to couple the first barrier layer 524a and the second barrier layer 524b to the leaflet 212. In some examples, the pledget 534 can extend through the holes 566 of the second barrier layer 524b to couple the leaflet 212 and the second barrier layer 524b together.

A portion of the second edge 532a of the first barrier layer 524a can be axially aligned with the inflow end 208 of the frame 202, as shown in FIG. 15. In some examples, the second edge 532b of the second barrier layer 524b can extend axially beyond the inflow end 208 of the frame 202, and the second end portion 530b of the second barrier layer 524b can be wrapped around the inflow end of the frame 202 towards a radially outer surface of the frame 202.

In some examples, the first edge 528a of the first barrier layer 524a can have angled portions or a zig-zag pattern that corresponds with the angled struts 218 of the frame 212. For example, the first edge 528a of the first barrier layer 524a can be axially aligned with the struts 218 positioned at the inflow end of the outflow cells 215 of the frame 202. In some examples, the first barrier layer 524a can also include slits 568 (FIG. 16A) to facilitate easier wrapping of the first end portion 526a of the first barrier layer 524a around the struts 218 of the frame 202 and/or easier coupling to an outer skirt (e.g., outer skirt 506).

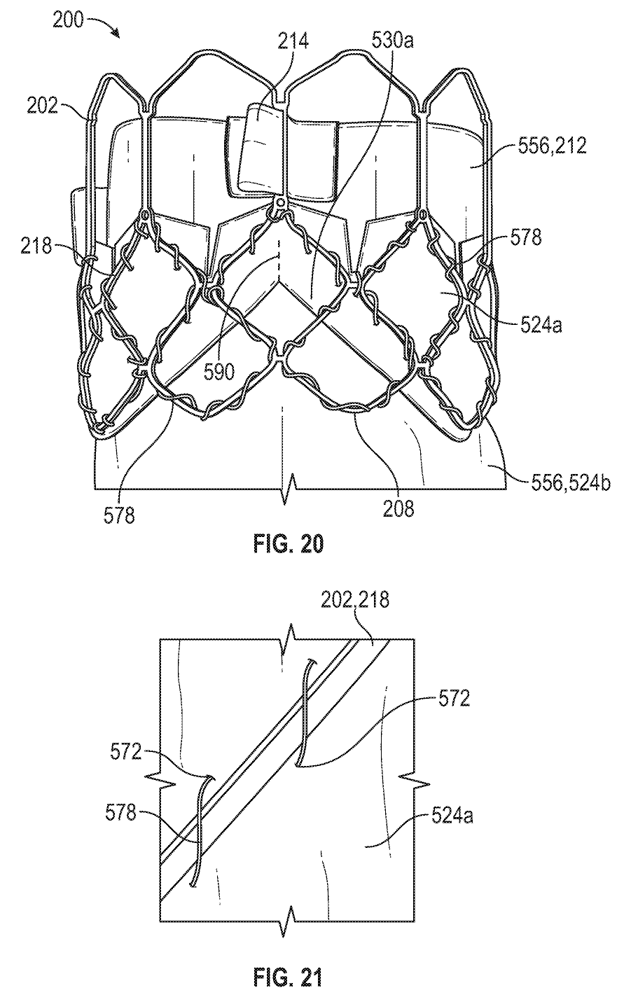

As shown in FIGS. 15–16B, each of the first barrier layer 524a and the second barrier layer 524b are shown as segments which extend around only a portion of the frame 202 in the circumferential direction. For example, each of the first barrier layer 524a and the second barrier layer 524b segments can correspond to one leaflet 212. In some examples, multiple of these segments may be coupled together at their sides to form barrier layers that extend continuously around the circumference of the frame 202. For example, the first barrier layer 524a and the second barrier layer 524b include angled side portions 570a, 570b respectively for connecting to other barrier layer segments. In some examples, the first barrier layer 524a and the second barrier layer 524b can be a single piece that extends the circumference of the frame 202. For example, FIG. 18 illustrates the second barrier layer 524b as a single piece that extends the length of three leaflets 212. FIG. 20 also illustrates a continuous first barrier layer 524a.

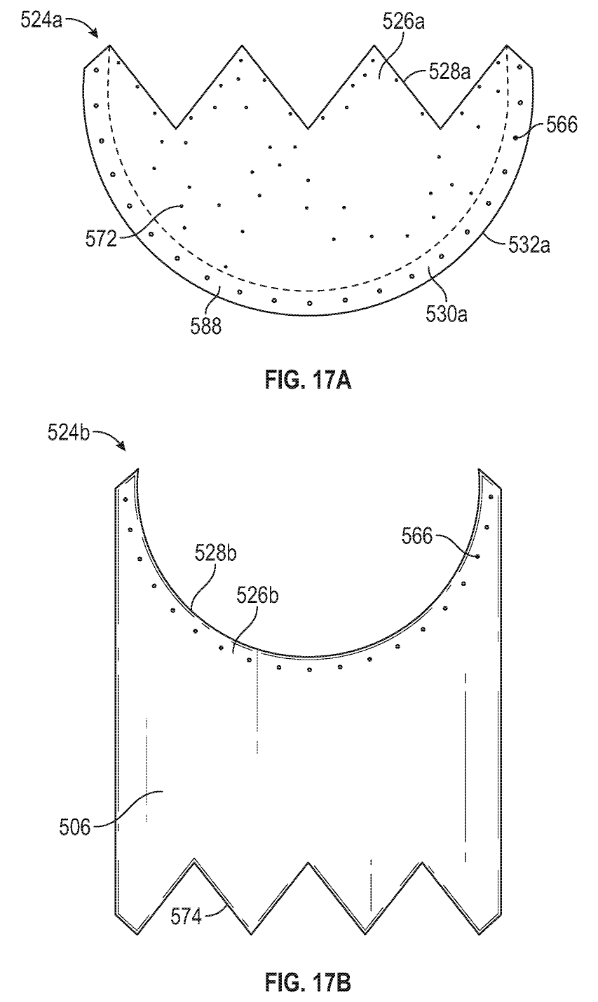

As shown in FIG. 17A, the first barrier layer 524a can include a plurality of holes 572, in some examples. The holes 572 are positioned throughout the body of the first barrier layer 524a, for example, between the holes 566 and the first edge 528a, such that the holes 572 are positioned adjacent to struts 212 of the frame 202 when the first barrier layer 524a is disposed within the frame 202. For example, as described in more detail below, fasteners (e.g., sutures) can pass through the holes 572 to couple the first barrier layer 524a to the frame 202, in some examples. The holes 572 can be omitted from the body of the first barrier layer 524a in the “suture-less” approach (see FIG. 16A).

As shown in FIG. 17B, the outer skirt 506 can be integrated with the second barrier layer 524b. The outer skirt 506 is configured to be wrapped or folded around the inflow end 208 of the frame 202, such that the outer skirt 506 is positioned radially outwards of the frame 202 and an outflow edge 574 of the outer skirt 506 is positioned towards the outflow end 210 of the frame 202. The outflow edge 574 of the outer skirt 506 can have angled portions or a zig-zag pattern that corresponds with the angled struts 218 of the frame 212. For example, the outflow edge 574 can be axially aligned with the struts 218 positioned at the inflow end of the outflow cells 215 of the frame 202. In some examples, the outer skirt 506 can also include slits 568 (FIG. 18) to facilitate easier wrapping of the outflow edge 574 around the struts 218 of the frame 202 and/or easier coupling to the first barrier layer 524a.

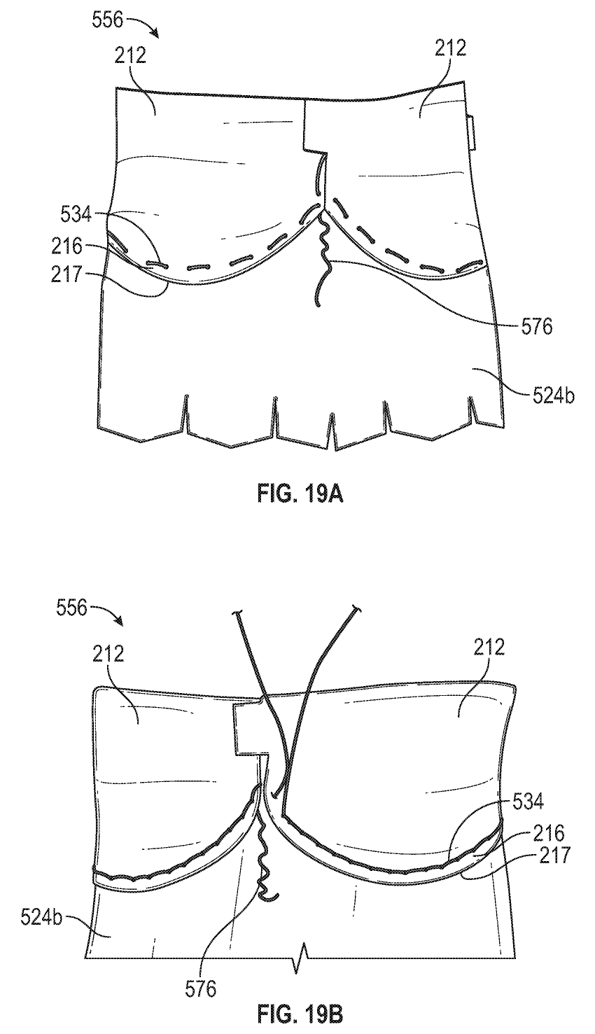

To assemble valve 200 with the first and second barrier layers 524a, 524b, the second barrier layer 524b is initially coupled to the leaflets 212. As shown in FIG. 18, to form the leaflet construct 556, the pledget 534 extends through the cusp edge portion 216 of the leaflets 212 and the first end portion 526b of the second barrier layer 524b in an in-and-out pattern to couple the second barrier layer 524b and the leaflets 212 together. In some examples, the pledget 534 can extend through the cusp edge portion 216 of the leaflet 212 and the first end portion 526b of the second barrier layer 524b in a single pass of the in-and-out pattern, as shown in FIGS. 18 and 19A. As such, the pledget 534 is visible and accessible on either side of the leaflet construct 556 in spaced apart or discontinuous segments of the pledget 534. As shown in FIG. 18, the second barrier layer 524b and leaflets 212 are initially in a laid flat configuration. After the second barrier layer 524b and leaflets 212 are coupled together with the pledget 534, the ends of the leaflet construct 556 are coupled together with fasteners 576 (e.g., sutures), such that the leaflet construct 556 is in a cylindrical shape, as shown in FIG. 19A.

In some examples, the pledget 534 can extend through the leaflet 212 and the second barrier layer 524b in a double pass of the in-and-out pattern. For example, the pledget 534 can extend through the cusp edge portion 216 and the first end portion 526b in an in-and-out pattern along a first path (e.g., starting on a radially outward side of the leaflet construct 556) and can also extend through the cusp edge portion 216 and the first end portion 526b in an in-and-out pattern along a second path that is opposite to the first path (e.g., starting on a radially inward side of the leaflet construct 556). In this way, the pledget 534 can be substantially continuous along both the radially inner and radially outer sides of the leaflet construct 556, as shown in FIG. 19B, and can provide more locations for other components to be coupled to either side of the leaflet construct 556.

As introduced above, the first barrier layer 524a can be coupled to the frame 202 with fasteners 578 (e.g., sutures) or in a suture-less approach. FIGS. 20–21 illustrate the first barrier layer 524a coupled to the frame 202 with fasteners 578 (e.g., sutures). For example, the fasteners 578 can pass through the holes 572 of the first barrier layer 524a and around the struts 218 of the frame 202 as whip stitches applied from the outside of the frame 202. As shown in FIG. 21, the holes 572 can be positioned in a staggered zig-zag pattern, such that the holes 572 are positioned on either side of a strut 218 of the frame 202 when the first barrier layer 524a is disposed within the frame 202. Directly attaching the first barrier layer 524a to the struts 218 in this manner can be simpler and faster than known methods, while also minimizing the suture area exposed inwardly of the first barrier layer 524a (e.g., between the first barrier layer 524a and the leaflets 212). To further minimize the suture area exposed inwardly of the first barrier layer 524a, the first barrier layer 524a can be coupled to the frame 202 in a suture-less manner such that the first barrier layer 524a and frame 202 are fused together (e.g., with adhesive 564, etc.) in some examples.

In some examples, as shown in FIG. 20, the fasteners 578 can also couple at least a portion of the second barrier layer 524b to the frame 202, for example, at locations where the second barrier layer 524b is directly adjacent to the frame 202 and where the first barrier layer 524a is not radially between the frame 202 and the second barrier layer 524b (e.g., adjacent to the inflow end 208 of the frame 202 and circumferentially aligned with the commissure 214).

In some examples, the first barrier layer 524a is coupled to the frame 202 prior to coupling the leaflet construct 556 to the frame 202 (e.g., in the suture-less approach). In some examples, the first barrier layer 524a is coupled to the leaflet construct 556 before being coupled to the frame 202. In some examples, as shown in FIG. 20, the leaflet construct 556 and the first barrier layer 524a can both be positioned within the frame 202 when the first barrier layer 524a is coupled to the frame 202 (e.g., with fasteners 578).

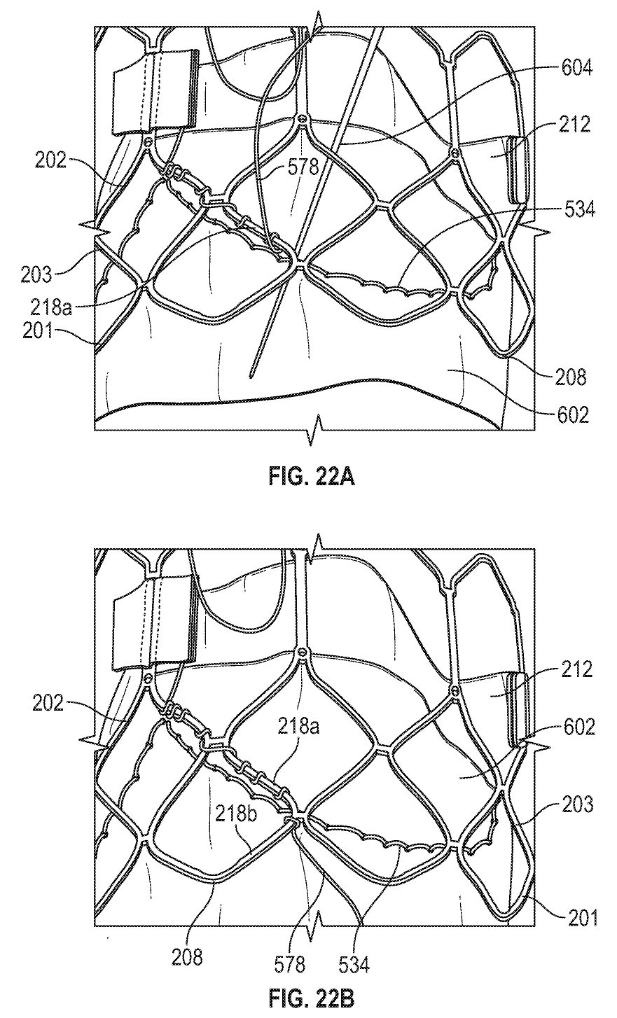

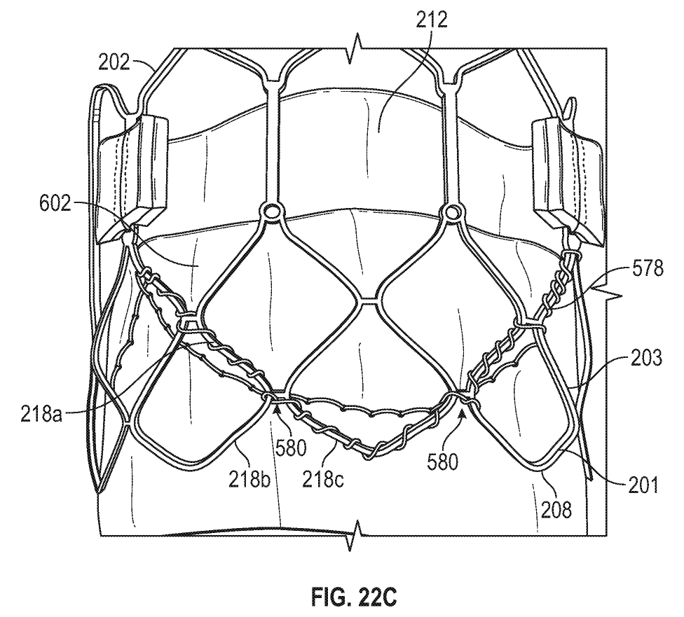

In some examples, when the fasteners 578 connecting the struts 218 to the first barrier layer 524a cross the cusp edge portion 216 of the leaflet 212, the fasteners 578 can catch the pledget 534 utilizing a hammock stitch to help secure the leaflet construct 556 to the frame 202. An example technique for forming a hammock stitch 580 is shown in FIGS. 22A–22C. For purposes of illustration, FIGS. 22A–22C illustrate the hammock technique with an inner skirt 602 that is coupled to the leaflets 212 with the pledget 534, rather than the first and second barrier layers 524a, 524b. As shown in FIG. 22A, at the junction where the fasteners 578 and the pledget 534 (e.g., and the cusp edge portion 216 of the leaflet 212) intersect, a needle 604 carrying the fasteners 578 is inserted between the pledget 534 and the inner skirt 602. In examples having the first and second barrier layers 524a, 524b, the needle 604 is passed through the first barrier layer 524a and radially between the pledget 534 and the leaflet 212. As shown in FIG. 22A, the needle 604 is inserted radially inwards through a cell of the frame 202 adjacent to a strut 218a of a second row 203 of struts 218 (e.g., between the strut 218a and the outflow end 210 of the frame 202). The second row 203 of struts 218 is adjacent to a first (or inflow) row 201 of struts 218 disposed at the inflow end 208 of the frame 202. The needle 604 is then passed between the pledget 534 and the inner skirt 602. The needle 604 is then brought radially outwards around the inflow end 208 of the frame 202.

The needle 604 carrying the fasteners 578 is then looped around a strut 218b of the inflow row 201 of struts 218 that is circumferentially aligned with the strut 218a, as shown in FIG. 22B. Thereafter, the needle 604 carrying the fasteners 578 is looped around a strut 218c of the inflow row 201 of struts 218 that is adjacent to the strut 218b to form a hammock stitch 580, as shown in FIG. 22C. Formation of the whip stitches continues around the strut 218c and the inner skirt 602 to the inflow end 208 of the frame 202 and then continues towards the outflow end of the skirt 602. When the fasteners 578 subsequently cross the cusp edge portion 216 of the same leaflet 212 for a second time, a similar hammock stitch 580 is formed through the pledget 534 and around the struts 218 of the frame 202 (e.g., around struts 218 of the first row 201 of struts 218), as shown in FIG. 22C.

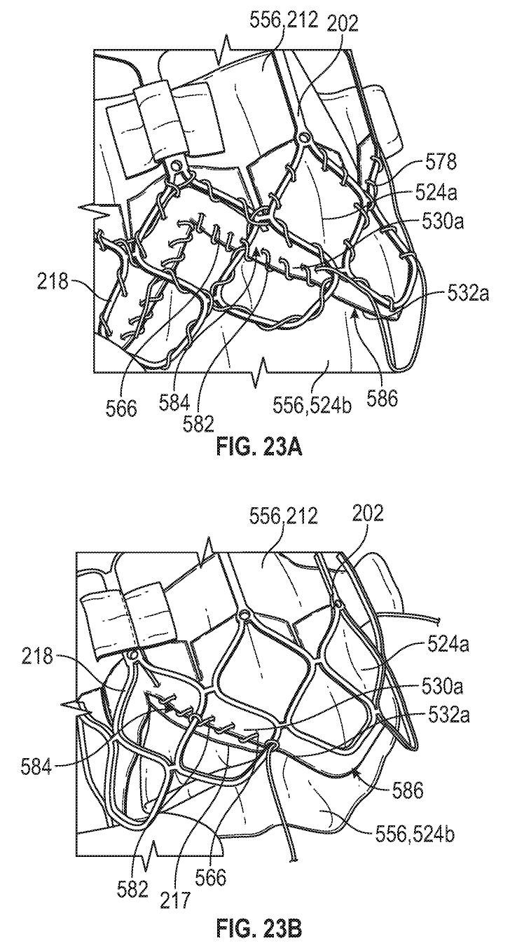

After the first barrier layer 524a is coupled to the frame 202, the second end portion 530a of the first barrier layer 524a is coupled to the leaflet construct 556. FIGS. 23A–23B illustrate the first barrier layer 524a partially coupled to the leaflet construct 556, with FIG. 23A depicting an approach that uses fasteners 578 and FIG. 23B depicting the “suture-less” approach (e.g., without fasteners 578). For example, as shown in FIG. 23A, fasteners 582 (e.g., sutures, etc.) can pass through the holes 566 of the first barrier layer 524a and whip around both the second edge 532a of the first barrier layer 524a and the cusp edge 217 of the leaflet 212 as whip stitches. In some examples, as shown in FIG. 23B, the fasteners 582 can pass through the holes 566 of the first barrier layer 524a and whip around the second edge 532a of the first barrier layer 524a and the pledget 534 as whip stitches, without surrounding or capturing the cusp edge 217 of the leaflet 212. As shown in FIG. 23B, the fasteners 582 which couple the first barrier layer 524a to the leaflet construct 556 can be looped around the frame 202 at locations where the second end portion 530a of the first barrier layer 524a and the pledget 534 intersect with struts 218 of the frame 202, in some examples.

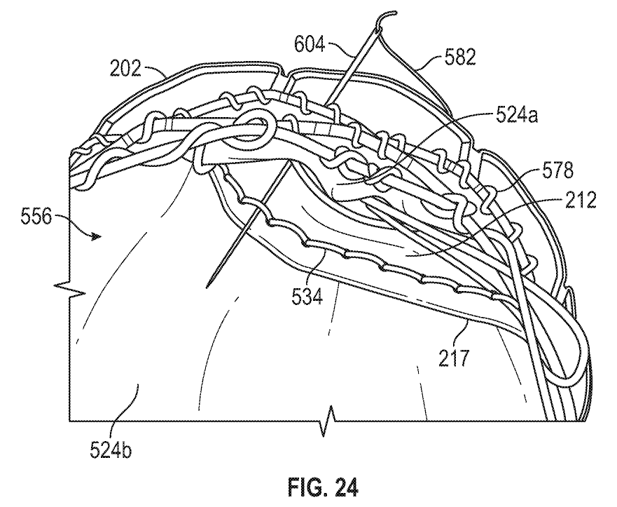

As described above, the holes 566 in the first barrier layer 524a follow the curved shape of the cusp edge portion 216 of the leaflet 212, rather than following the shape of the second end portion 530a of the first barrier layer 524a. In some examples, as shown in FIGS. 23A–23B, the first barrier layer 524a extends beyond the cusp edge 217 of the leaflet 212 towards the inflow end 208 of the frame 202 (see also FIG. 14), and in some examples, beyond the inflow end 208 of the frame 202. As such, the fasteners 582, which whip around the second edge 532a of the first barrier layer 524a along side portions 584, are not whipped around the second edge 532a of the first barrier layer 524a along an inflow portion 586 of the first barrier layer 524a. Instead, a needle 604 carrying the fasteners 582 is passed through the holes 566 of the first barrier layer 524a and between the pledget 534 and the leaflet 212, as shown in FIG. 24, such that the fasteners 582 loop around the pledget 534. In this way, a different stitch pattern can be used along the side portions 584 of the first barrier layer 524a than the inflow portion 586 of the first barrier layer 524a, when coupling the first barrier layer 524a to the leaflet construct 556.

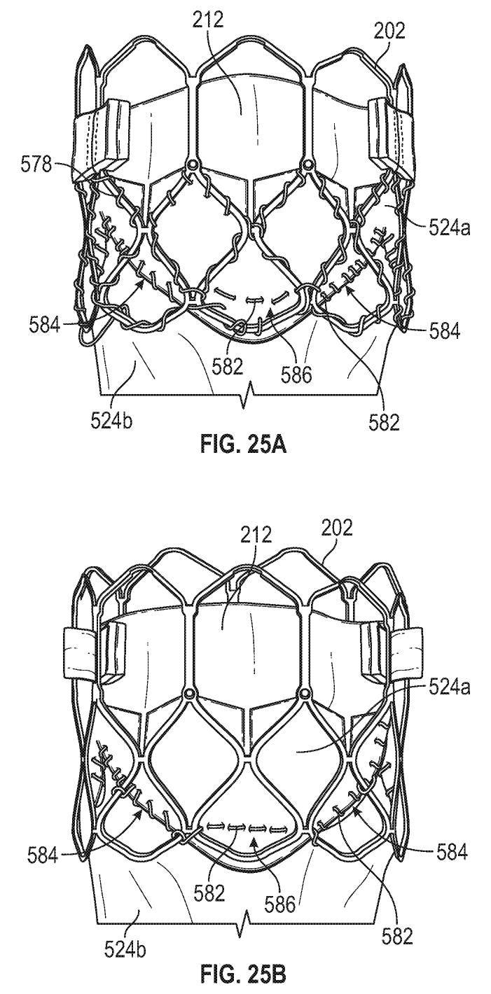

After the inflow portion 586 of the first barrier layer 524a is coupled to the leaflet construct 556, the fasteners 582 are coupled along the second side portion 584 of the first barrier layer 524a in the same manner as the first side portion 584, as shown in FIGS. 25A–25B. FIGS. 25A–25B illustrate the first barrier layer 524a coupled to the leaflet construct 556, with FIG. 25B depicting the “suture-less” approach (e.g., without fasteners 578).

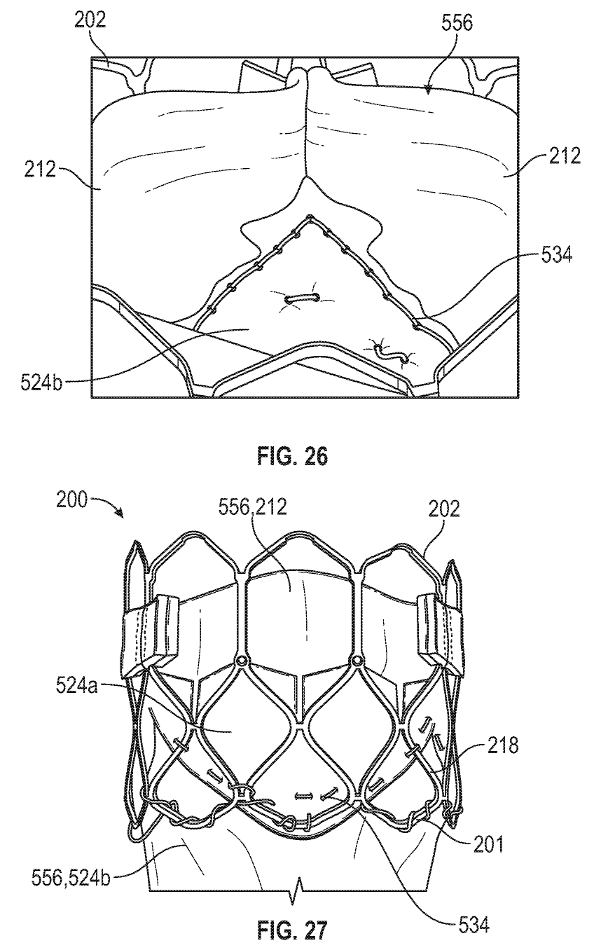

In some examples, the first barrier layer 524a is coupled to the leaflet construct 556 via the pledget 534, instead of with fasteners 582. For example, the pledget 534 can extend in an in-and-out pattern through the second end portion 530a of the first barrier layer 524a, the cusp edge portion 216 of the leaflet 212, and the first end portion 526b of the second barrier layer 524b. In some examples, the cusp edge portion 216 of the leaflet 212 and the first end portion 526b of the second barrier layer 524b can initially be coupled together with a single pass of the pledget 534 in an in-and-out pattern, as shown in FIGS. 18 and 19A. The second end portion 530a of the first barrier layer 524a can be coupled to the leaflet construct 556 by a second, single pass of the pledget 534. In this way, the pledget 534 is passed through the leaflet 212 and second barrier layer 524b twice (e.g., double-passed) and through the first barrier layer 524a once (e.g., single pass). As such, when viewed from an interior of the frame 202 as shown in FIG. 26, the pledget 534 forms a continuous, double-passed pledget line. FIG. 27 illustrates the valve 200 from the exterior of the frame 202 and as shown the pledget 534 forms a single-passed pledget line having spaced apart segments.

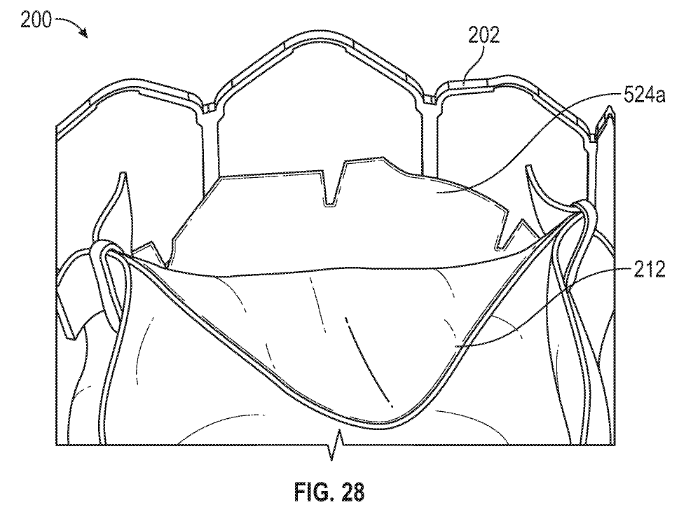

In some examples, the first barrier layer 524a can be coupled to the frame 202 prior to coupling the first barrier layer 524a to the leaflet construct 556 with the pledget 534. As such, the pledget 534 can extend around a radially outer surface of one or more struts 218 of the frame 202 when coupling the first barrier layer 524a to the leaflet construct 556, as shown in FIG. 27. In some examples, as shown, the first barrier layer 524a can be fused to the frame 202 with the adhesive 564 (FIG. 14). This “suture-less” approach for coupling the first barrier layer 524a to the frame 202 can result in fewer sutures or stitches that are positioned radially between the leaflet 212 and the first barrier layer 524a, which can prevent or reduce abrasion between the leaflet 212 and the sutures during coaptation of the leaflets 212. For example, as shown in FIG. 28, there are no sutures positioned radially between the first barrier layer 524a and the leaflet 212 in the suture-less approach.

In some examples, the pledget 534 can be used to couple struts 218 of the first row 201 of struts 218 to the second barrier layer 524b and/or to the first barrier layer 524a. In some examples, a separate fastener (e.g., sutures) can be used to couple the first row 201 of struts 218 to the second barrier layer 524b and/or to the first barrier layer 524a.

In some examples, the first barrier layer 524a can comprise a material that is substantially opaque, which can make it difficult to see the leaflet construct 556 when coupling the first barrier layer 524a to the leaflet construct 556. In some examples, to improve ability to see the leaflet construct 556 when fastening from the outside of the frame 202, a portion of the first barrier layer 524a can be heat treated (e.g., pressed or flattened) such that the portion is transparent enough to allow visibility of the pledget 534 along the cusp edge portion 216 of the leaflet 212. For example, as shown in FIG. 17A, a portion 588 of the first barrier layer 524a (e.g., between the dashed line and the second edge 532a) can be heat treated, such that the portion 588 is sufficiently transparent.

In some examples, to improve visibility of the leaflet construct 556 when coupling the first barrier layer 524a to the leaflet construct 556, the first barrier layer 524a can include slits 590 which extend from the second end portion 530a to the second edge 532a, as shown in FIG. 20. In some examples, the slits 590 are circumferentially aligned with the commissures 214. The slits 590 enable the second end portion 530a of the first barrier layer 524a to be manipulated or moved out of the way to improve visibility of the leaflet construct 556 during coupling.

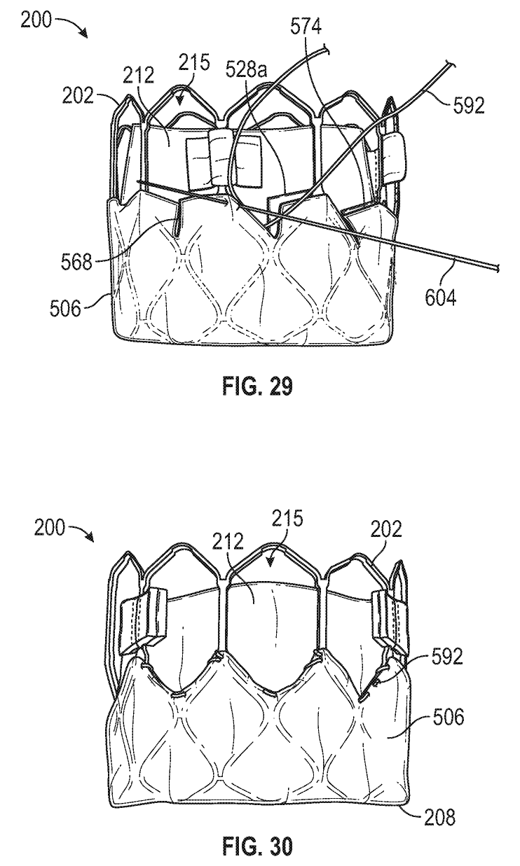

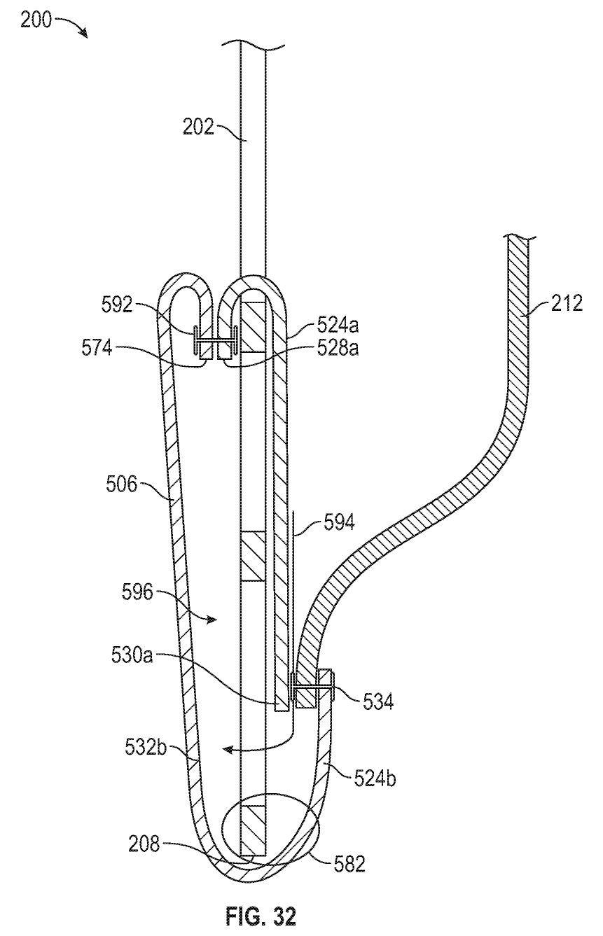

After the leaflet construct 556 and the first barrier layer 524a are coupled together, an outer skirt can be coupled to an outer surface of the frame 202. In some examples, the outer skirt can be a separate outer skirt (e.g., outer skirt 106, outer skirt 206). In some examples, the outer skirt can be integrated with the second barrier layer 524b (e.g., outer skirt 506). As shown in FIG. 29, the outer skirt 506, which is integrated with the second barrier layer 524b, is wrapped around the inflow end 208 of the frame 202 to the outer surface of the frame 202. The outflow edge 574 of the outer skirt 506 can be axially aligned with the first edge 528a of the first barrier layer 524a, such that the zig-zag patterns align. The first barrier layer 524a and the outer skirt 506 can be coupled together with fasteners 592 (e.g., sutures) adjacent to the inflow end of the outflow cells 215 of the frame 202. In some examples, flaps formed adjacent to the slits 568 can be folded together prior to coupling the outer skirt 506 to the first barrier layer 524a, such that the flaps are positioned between the outer skirt 506 and the first barrier layer 524a (see also FIG. 32). As shown in FIG. 30, after the outer skirt 506 and the first barrier layer 524a are coupled together, the outer skirt 506 of the prosthetic valve 200 extends in the axial direction from the inflow end 208 of the frame 202 to the inflow end of the outflow cells 215.

In some examples, as shown in FIG. 31, the leaflet 212 can be folded such that the cusp edge 217 is positioned towards the outflow end of the frame 202, rather than towards the inflow end 208 of the frame 202 (e.g., FIG. 14). As shown in FIG. 31, the first and second barrier layers 524a, 524b are coupled to the pledget 534 with fasteners 582 (e.g., the pledget 534 extends through only the leaflet 212). In some examples, the pledget 534 can extend through the leaflet 212 and the second barrier layer 524b, as described above. In some examples, folding the leaflet 212 in this manner can allow for easier attachment of the first barrier layer 524a to the leaflet 212, as the fasteners 582 coupling the first barrier layer 524a to the leaflet 212 are passed around the pledget 534 on the rough side of the leaflet 212, instead of on the smooth side of the leaflet 212.

As shown in FIG. 32, it can be desirable in some examples for blood to flow in between the first barrier layer 524a and the leaflet 212 along path 594 during operation of the valve 200, for example, during diastole. In particular, during diastole, blood can enter a pocket 596 formed between the outer skirt 506, the first barrier layer 524a, and the second barrier layer 524b, inflating the pocket 596. This can contribute to improved sealing of by inflating the pocket 596 against the native anatomy. As the pocket 596 inflates, blood is retained within the pocket 596 in part by the folded over flaps of the outer skirt 506 and the first barrier layer 524a. As shown in FIG. 32, the outflow edge 574 of the outer skirt 506 and the first edge 528a of the first barrier layer 524a are folded over towards each other and coupled together with fasteners 592. In some examples, the second end portion 530a of the first barrier layer 524a can function as a valve (e.g., a duckbill valve) to further retain the blood within the pocket 596.

In some examples, the technique used to couple the first barrier layer 524a to the leaflet construct 556 can allow for a looser connection between the first barrier layer 524a and the leaflet 212, such that more blood is permitted to enter the pocket 596 along the path 594. For example, some of the techniques described herein, such as using a single pass of the pledget 534 as described above in connection with FIGS. 26–27, can allow more blood to enter the pocket 596. In some examples, to further increase the ability of blood to flow into the pocket 596, the first barrier layer 524a can be coupled to the leaflet construct 556 with hammock stitches 580 only at the locations where the pledget 534 cross the struts 218 of the frame 202, rather than having fasteners 582 extend along the length of the side portions 584 and inflow portion 586 of the first barrier layer 524a. As shown in FIG. 32, fasteners 598 (e.g., sutures) couple the second barrier layer 524b to the inflow end 208 of the frame 202.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame having an inflow end and an outflow end; a plurality of leaflets disposed within the annular frame, each leaflet comprising a cusp edge portion that is positioned towards the inflow end of the frame; a skirt coupled to the annular frame; and a barrier layer coupled to each leaflet and comprising a first end and a second end, wherein the first end is coupled to the cusp edge portion and the second end is coupled to the skirt, wherein the barrier layer at least partially extends around the inflow end of the annular frame.

Example 2. The prosthetic heart valve of any example herein, particularly example 1, wherein the barrier layer comprises a material that is resistant to tissue ingrowth.

Example 3. The prosthetic heart valve of any example herein, particularly either example 1 or example 2, wherein the barrier layer comprises an intermediate portion positioned between the first end and the second end, and wherein the intermediate portion at least partially extends around the inflow end of the annular frame.

Example 4. The prosthetic heart valve of any example herein, particularly example 3, wherein the first end of the barrier layer comprises a fold and a free edge, wherein the free edge is positioned between the intermediate portion of the barrier layer and the annular frame.

Example 5. The prosthetic heart valve of any example herein, particularly any one of examples 1–4, wherein the first end of the barrier layer is coupled to the cusp edge portion with first sutures.

Example 6. The prosthetic heart valve of any example herein, particularly any one of examples 1–5, wherein the second end of the barrier layer is coupled to the skirt with second sutures.

Example 7. The prosthetic heart valve of any example herein, particularly any one of examples 1–6, wherein a first distance between the inflow end of the frame and the first end of the barrier layer at a location closest to the outflow end is within the range of 0.5 mm to 3 mm.

Example 8. The prosthetic heart valve of any example herein, particularly any one of examples 1–7, wherein a second distance between the inflow end of the frame and the second end of the barrier layer at a location closest to the outflow end is within the range of 3 mm to 10 mm.

Example 9. The prosthetic heart valve of any example herein, particularly any one of examples 1–8, wherein a third distance between the inflow end of the frame and an inflow end of the skirt is within the range of 0.5 mm to 2 mm.

Example 10. The prosthetic heart valve of any example herein, particularly any one of examples 1–9, further comprising an inner skirt coupled to an interior of the annular frame.

Example 11. The prosthetic heart valve of any example herein, particularly any one of examples 1–10, wherein the barrier layer includes pores that are within the range of 0.5 µm to 20 µm.

Example 12. The prosthetic heart valve of any example herein, particularly any one of examples 1–10, wherein the barrier layer includes pores that are within the range of 20 µm to 200 µm.

Example 13. The prosthetic heart valve of any example herein, particularly any one of examples 1–10, wherein the barrier layer comprises a polymeric material that is impermeable to fluids.

Example 14. A prosthetic heart valve comprising: an annular frame; a plurality of leaflets disposed within the annular frame, each leaflet comprising a cusp edge portion comprising a suture line and a free edge; and a barrier layer coupled to a cusp edge portion of each leaflet at the suture line, wherein the free edge of the leaflet is positioned between the barrier layer and the frame.

Example 15. The prosthetic heart valve of any example herein, particularly example 14, wherein the barrier layer comprises a polymeric material that is resistant to tissue ingrowth.

Example 16. The prosthetic heart valve of any example herein, particularly example 15, wherein the barrier layer comprises a suture extending along a length of the barrier layer, wherein the polymeric material surrounds a circumference of the suture.

Example 17. The prosthetic heart valve of any example herein, particularly either example 15 or example 16, wherein the polymeric material comprises one or more folds.

Example 18. The prosthetic heart valve of any example herein, particularly any one of examples 14–17, further comprising fasteners that couple the barrier layer to the cusp edge portions of the leaflets.

Example 19. The prosthetic heart valve of any example herein, particularly any one of examples 14–18, further comprising an outer skirt disposed around an outer surface of the annular frame.

Example 20. The prosthetic heart valve of any example herein, particularly example 19, wherein the outer skirt comprises a first layer and a second layer, wherein a portion of the barrier layer is positioned axially between the first layer and the second layer.

Example 21. A leaflet construct comprising: a plurality of leaflets, each leaflet comprising a cusp edge portion; a barrier layer coupled to the cusp edge portion of each leaflet, the barrier layer comprising a polymeric layer that is resistant to tissue ingrowth and a suture extending through the polymeric layer.

Example 22. The leaflet construct of any example herein, particularly example 21, wherein the suture is a 3-0 suture.

Example 23. The leaflet construct of any example herein, particularly either example 21 or example 22, further comprising fasteners that couple the barrier layer to the leaflet.

Example 24. A prosthetic heart valve comprising: an annular frame having an inflow end and an outflow end; a plurality of leaflets disposed within the annular frame, each leaflet comprising a cusp edge that is positioned towards the inflow end of the frame; and a barrier layer disposed within the annular frame, wherein at least a portion of the cusp edge of the leaflet is positioned between the barrier layer and the frame.

Example 25. The prosthetic heart valve of any example herein, particularly example 24, wherein the barrier layer at least partially extends around the inflow end of the frame.

Example 26. The prosthetic heart valve of any example herein, particularly either example 24 or example 25, further comprising an outer skirt disposed around an outer surface of the annular frame and having an inflow edge, wherein the outer skirt at least partially extends around the inflow end of the frame, and wherein the inflow edge of the outer skirt is disposed radially inwardly of the frame.

Example 27. A prosthetic heart valve comprising: an annular frame having an inflow end, an outflow end, and a central longitudinal axis extending from the inflow end to the outflow end; a plurality of leaflets disposed within the annular frame, each leaflet comprising a cusp edge that is positioned towards the inflow end of the frame; a first barrier layer coupled to the frame, wherein the first barrier layer is radially positioned between the leaflets and the frame; and a second barrier layer disposed within the annular frame and comprising a first end and a second end, wherein at least a portion of the cusp edge of the leaflet is radially positioned between the first end of the second barrier layer and the frame, and wherein the second end of the second barrier layer is positioned towards the inflow end of the annular frame.

Example 28. The prosthetic heart valve of any example herein, particularly example 27, wherein the second end of the second barrier layer is coupled to an inner surface of the frame.

Example 29. The prosthetic heart valve of any example herein, particularly example 27, wherein the second barrier layer at least partially extends around the inflow end of the frame.

Example 30. The prosthetic heart valve of any example herein, particularly any one of examples 27–29, further comprising an outer skirt disposed around an outer surface of the annular frame.

Example 31. The prosthetic heart valve of any example herein, particularly example 30, wherein the outer skirt is integrated with the second barrier layer.

Example 32. The prosthetic heart valve of any example herein, particularly any one of examples 27–31, wherein the second barrier layer is positioned closer to the inflow end of the frame than the first barrier layer.

Example 33. The prosthetic heart valve of any example herein, particularly any one of examples 27–32, wherein the first end of the second barrier layer is a free end.

Example 34. The prosthetic heart valve of any example herein, particularly any one of examples 27–33, wherein an inflow edge of the first barrier layer is curved, and wherein an edge at the first end of the second barrier layer is curved.

Example 35. The prosthetic heart valve of any example herein, particularly example 30, wherein the outer skirt and the second barrier layer are a single piece.

Example 36. The prosthetic heart valve of any example herein, particularly any one of examples 27–35, further comprising a pledget, wherein the plurality of leaflets and the first end of the second barrier layer are coupled together by the pledget.

Example 37. The prosthetic heart valve of any example herein, particularly example 36, wherein the first barrier layer is coupled to the frame with adhesive.

Example 38. The prosthetic heart valve of any example herein, particularly example 36, wherein the first barrier layer is coupled to the frame with fasteners.

Example 39. The prosthetic heart valve of any example herein, particularly example 38, wherein the first barrier layer includes holes in a zig-zag pattern adjacent to struts of the frame, wherein the fasteners extend through the holes of the first barrier layer and around the struts of the frame.

Example 40. The prosthetic heart valve of any example herein, particularly either example 38 or example 39, wherein the fasteners extend around the frame and the pledget at locations where the pledget intersects with a strut of the frame.

Example 41. The prosthetic heart valve of any example herein, particularly example 40, wherein the fasteners extend around the frame and the pledget and loop around a strut of an inflow row of struts to form a hammock stitch at the locations where the pledget intersects with a strut of the frame.

Example 42. The prosthetic heart valve of any example herein, particularly any one of examples 36–41, wherein the first barrier layer is coupled to the plurality of leaflets with fasteners, wherein the fasteners extend around an edge of the first barrier layer along side portions of the first barrier layer and wherein the fasteners do not extend around the edge of the first barrier layer along an inflow portion of the first barrier layer.

Example 43. The prosthetic heart valve of any example herein, particularly any one of examples 36–41, wherein the plurality of leaflets, the first end of the second barrier layer, and the first barrier layer are coupled together by the pledget.

Example 44. The prosthetic heart valve of any example herein, particularly any one of examples 27–43, wherein a pocket is formed between the first barrier layer and the second barrier layer, wherein blood is permitted to flow into the pocket during diastole whereby the pocket is expanded against native anatomy.

The features described herein with regard to any example can be combined with other features described in any one or more of the other examples, unless otherwise stated. For example, any one or more of the features of one outer skirt for a prosthetic heart valve can be combined with any one or more features of another outer skirt for a prosthetic heart valve. As another example, any one or more features of one barrier layer for a prosthetic heart valve can be combined with any one or more features of another barrier layer for a prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame having an inflow end and an outflow end;
a plurality of leaflets disposed within the annular frame, each leaflet comprising a cusp edge portion that is positioned towards the inflow end of the frame;
a skirt coupled to the annular frame; and
a barrier layer coupled to each leaflet and comprising a first end and a second end, wherein the first end is coupled to the cusp edge portion and the second end is coupled to the skirt, wherein the barrier layer at least partially extends around the inflow end of the annular frame.

2. The prosthetic heart valve of claim 1, wherein the barrier layer comprises a material that is resistant to tissue ingrowth.

3. The prosthetic heart valve of claim 1, wherein the barrier layer comprises an intermediate portion positioned between the first end and the second end, and wherein the intermediate portion at least partially extends around the inflow end of the annular frame.

4. The prosthetic heart valve of claim 3, wherein the first end of the barrier layer comprises a fold and a free edge, wherein the free edge is positioned between the intermediate portion of the barrier layer and the annular frame.

5. The prosthetic heart valve of claim 1, wherein the first end of the barrier layer is coupled to the cusp edge portion with first sutures, and wherein the second end of the barrier layer is coupled to the skirt with second sutures.

6. The prosthetic heart valve of claim 1, wherein the barrier layer comprises a polymeric material and a suture extending along a length of the barrier layer, wherein the polymeric material surrounds a circumference of the suture.

7. The prosthetic heart valve of claim 1, wherein the skirt comprises a first layer and a second layer, wherein a portion of the barrier layer is positioned axially between the first layer and the second layer.

8. The prosthetic heart valve of claim 1, wherein the skirt at least partially extends around the inflow end of the frame, and wherein an inflow edge of the skirt is disposed radially inwardly of the frame.

9. A prosthetic heart valve comprising:
an annular frame having an inflow end and an outflow end;
a plurality of leaflets disposed within the annular frame, each leaflet comprising a cusp edge that is positioned towards the inflow end of the frame; and
a barrier layer disposed within the annular frame, wherein at least a portion of the cusp edge of the leaflet is positioned between the barrier layer and the frame.

10. The prosthetic heart valve of claim 9, wherein the barrier layer at least partially extends around the inflow end of the frame.

11. The prosthetic heart valve of claim 9, further comprising an outer skirt disposed around an outer surface of the annular frame and having an inflow edge, wherein the outer skirt at least partially extends around the inflow end of the frame, and wherein the inflow edge of the outer skirt is disposed radially inwardly of the frame.

12. A prosthetic heart valve comprising:
an annular frame having an inflow end, an outflow end, and a central longitudinal axis extending from the inflow end to the outflow end;
a plurality of leaflets disposed within the annular frame, each leaflet comprising a cusp edge that is positioned towards the inflow end of the frame;
a first barrier layer coupled to the frame, wherein the first barrier layer is radially positioned between the leaflets and the frame; and
a second barrier layer disposed within the annular frame and comprising a first end and a second end, wherein at least a portion of the cusp edge of the leaflet is radially positioned between the first end of the second barrier layer and the frame, and wherein the second end of the second barrier layer extends axially towards the inflow end of the annular frame.

13. The prosthetic heart valve of claim 12, wherein the second end of the second barrier layer is coupled to an inner surface of the frame.

14. The prosthetic heart valve of claim 12, wherein the second barrier layer at least partially extends around the inflow end of the frame.

15. The prosthetic heart valve of claim 12, wherein an inflow edge of the first barrier layer is curved, and wherein an edge at the first end of the second barrier layer is curved.

16. The prosthetic heart valve of claim 12, further comprising an outer skirt, wherein the outer skirt and the second barrier layer are a single piece.

17. The prosthetic heart valve of claim 12, further comprising a pledget, wherein the plurality of leaflets and the first end of the second barrier layer are coupled together by the pledget.

18. The prosthetic heart valve of claim 17, wherein the first barrier layer is coupled to the frame with fasteners, wherein the fasteners extend around the frame and the pledget at locations where the pledget intersects with a strut of the frame.

19. The prosthetic heart valve of claim 17, wherein the first barrier layer is coupled to the plurality of leaflets with fasteners, wherein the fasteners extend around an edge of the first barrier layer along side portions of the first barrier layer and wherein the fasteners do not extend around the edge of the first barrier layer along an inflow portion of the first barrier layer.

20. The prosthetic heart valve of claim 17, wherein the plurality of leaflets, the first end of the second barrier layer, and the first barrier layer are coupled together by the pledget.
